(19) United States
(12) Patent Application Publication
(10) Pub. No.: US 2023/0309795 A1
(43) Pub. Date: Oct. 5, 2023

(54) ENT GUIDE SHAFT WITH DEFLECTABLE TIP AND DISTAL ENDOSCOPE CAP

(71) Applicants: Acclarent, Inc., Irvine, CA (US); Biosense Webster (Israel) Ltd., Yokneam (IL)

(72) Inventors: Carly N. Harad, Irvine, CA (US); Alex Gonzalez, Lake Forest, CA (US); Matthew S. Corbin, Santa Clara, CA (US); Shubhayu Basu, Anaheim, CA (US); Marc Dean, Fort Worth, TX (US); Cesar Fuentes-Ortega, Pasadena, CA (US); Shmuel Auerbach, Kerem Maharal (IL); Gigi Koe, Anaheim, CA (US); Raymond Yue-sing Tang, Rosemead, CA (US); Kokou A. Amefia, Aliso Viejo, CA (US); Erica E. Lovejoy, La Puente, CA (US)

(21) Appl. No.: 18/115,310

(22) Filed: Feb. 28, 2023

Related U.S. Application Data
(60) Provisional application No. 63/325,791, filed on Mar. 31, 2022.
(60) Provisional application No. 63/392,924, filed on Jul. 28, 2022.

(51) Int. Cl. A61B 1/00; A61B 1/06; A61B 1/05; A61B 1/07; A61B 5/06; A61B 1/12; A61B 1/018; A61B 18/14

(52) U.S. Cl. CPC A61B 1/00101; A61B 1/0655; A61B 1/00091; A61B 1/051; A61B 1/07; A61B 5/065; A61B 1/00128; A61B 1/126; A61B 1/018; A61B 18/1492; A61B 2018/00982

(57) ABSTRACT

An apparatus includes a shaft assembly and a distal endoscope cap. A flexible distal portion of the shaft assembly is laterally deflectable relative to a rigid proximal portion of the shaft assembly. The shaft assembly defines a working channel sized and configured to enable advancement of a working element. The distal endoscope cap is configured to attach to the distal end of the shaft assembly. The distal endoscope cap has an outer diameter that is larger than the outer diameter of the flexible distal portion. The distal endoscope cap includes a body having at least one coupling member for attaching the distal endoscope cap to the distal end of the shaft assembly, at least one image sensor secured to the body for visualizing an anatomical structure, and at least one illuminating element secured to the body for illuminating a field of view of the at least one image sensor.

### PRIORITY

This application claims the benefit of U.S. Pat. App. No. 63/392,924, entitled “ENT Guide Shaft with Deflectable Tip and Distal Endoscope Cap,” filed Jul. 28, 2022, the disclosure of which is incorporated by reference herein; and U.S. Pat. App. No. 63/325,791, entitled “ENT Guide Shaft with Deflectable Tip and Distal Endoscope Cap,” filed Mar. 31, 2022, the disclosure of which is incorporated by reference herein.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus.

It may also be desirable to ablate tissue within the ear, nose, or throat of a patient. For instance, such ablation may be desirable to remodel tissue (e.g., to reduce the size of a turbinate), to provide denervation (e.g., to disable the posterior nasal nerve), and/or for other purposes. Some such ablation treatments may include radiofrequency (RF) ablation with alternating current (AC) electrical energy; and/or irreversible electroporation (IRE) via pulsed field direct current (DC) electrical energy. To achieve ablation, an end effector with one or more needle electrodes or other kind(s) of tissue contacting electrodes may be activated with monopolar or bipolar electrical energy. Such ablation procedures may be carried out in conjunction with a dilation procedure or separately from a dilation procedure.

It may also be desirable to provide easily controlled placement of a dilation catheter, ablation instrument, or other ENT instrument in an anatomical passageway, including in procedures that will be performed only by a single operator. While several systems and methods have been made and used to position an ENT instrument in an anatomical passageway, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an example of a surgery navigation system being used on a patient seated in an example of a medical procedure chair;

FIG. 2A depicts a front perspective view of an example of an instrument with a slider in a proximal position, such that a working element shaft of the instrument is retracted proximally relative to an open distal end of a shaft assembly of the instrument;

FIG. 2B depicts a front perspective view of the instrument of FIG. 2A, with the slider in a distal position, such that the working element shaft is extended distally relative to the open distal end of the shaft assembly;

FIG. 3 depicts a front perspective view of a distal portion of the instrument of FIG. 2A, with an example of a distal endoscope cap removably attached to the open distal end of the shaft assembly;

FIG. 4 depicts a rear perspective view of the distal portion of the instrument of FIG. 2A, with the distal endoscope cap of FIG. 3 removably attached to the open distal end of the shaft assembly;

FIG. 5 depicts a front elevation view of the distal endoscope cap of FIG. 3;

FIG. 6 depicts a rear elevation view of the distal endoscope cap of FIG. 3;

FIG. 7 depicts a top cross-sectional view of the distal endoscope cap of FIG. 3, taken along line 7-7 in FIG. 6, showing the flow direction of irrigation fluid therethrough;

FIG. 8 depicts an exploded front elevation view of another example of a distal endoscope cap for use with the instrument of FIG. 2A and having a body with a two-piece construction;

FIG. 9 depicts a top perspective view of a lower body portion of the distal endoscope cap of FIG. 8;

FIG. 10 depicts a bottom perspective view of an upper body portion of the distal endoscope cap of FIG. 8;

FIG. 11 depicts an exploded front elevation view of another example of a distal endoscope cap for use with the instrument of FIG. 2A and having a body with a three-piece construction including a front plate portion;

FIG. 12 depicts a top perspective view of a lower body portion of the distal endoscope cap of FIG. 11;

FIG. 13 depicts a bottom perspective view of an upper body portion of the distal endoscope cap of FIG. 11;

FIG. 14 depicts a front perspective view of the front plate portion of the distal endoscope cap of FIG. 11;

FIG. 15 depicts a front perspective view of another example of a distal endoscope cap for use with the instrument of FIG. 2A and having a body with a three-piece construction including a front plug portion;

FIG. 16 depicts a top perspective view of a lower body portion of the distal endoscope cap of FIG. 15;

FIG. 17 depicts a bottom perspective view of an upper body portion of the distal endoscope cap of FIG. 15;

FIG. 18 depicts a front perspective view of the front plug portion of the distal endoscope cap of FIG. 15;

FIG. 19 depicts a rear perspective view of the front plug portion of FIG. 18;

FIG. 20 depicts a front perspective view of another example of a distal endoscope cap for use with the instrument of FIG. 2A and having a coupling collar;

FIG. 21 depicts a rear perspective view of the distal endoscope cap of FIG. 20;

FIG. 22 depicts a side cross-sectional view of the distal endoscope cap of FIG. 20 aligned with another example of a flexible shaft portion for positioning the coupling collar of the distal endoscope cap within a counterbore of the flexible shaft portion;

FIG. 23 depicts a front perspective view of another example of a distal endoscope cap for use with the instrument of FIG. 2A and having a single camera, a single illuminating element, and two navigation sensors;

FIG. 24 depicts a rear perspective view of the distal endoscope cap of FIG. 23;

FIG. 25 depicts a front perspective view of another example of a distal endoscope cap for use with the instrument of FIG. 2A and having two cameras, a single illuminating element, and two irrigation fluid conduit grooves;

FIG. 26 depicts a rear perspective view of the distal endoscope cap of FIG. 25;

FIG. 27 depicts a front perspective view of an example of a dual-lumen routing device for directing irrigation fluid conduits, wires, and/or optical fibers along the shaft assembly of the instrument of FIG. 2A;

FIG. 28 depicts a front perspective view of an example of a triple-lumen routing device for directing irrigation fluid conduits, wires, and/or optical fibers along the shaft assembly of the instrument of FIG. 2A;

FIG. 29A depicts a front perspective view of another example of an instrument, with a working element shaft of the instrument retracted proximally relative to an open distal end of a shaft assembly of the instrument;

FIG. 29B depicts a front perspective view of the instrument of FIG. 29A, with the working element shaft extended distally relative to the open distal end of the shaft assembly;

FIG. 30A depicts a rear perspective view of the instrument of FIG. 29A, with a strain relief of a handle assembly of the instrument in a first orientation about a longitudinal axis of the shaft assembly;

FIG. 30B depicts a rear perspective view of the instrument of FIG. 29A, with the strain relief of the handle assembly of the instrument in a second orientation about the longitudinal axis of the shaft assembly;

FIG. 31 depicts a disassembled front perspective view of the instrument of FIG. 29A, showing a linear actuator of the instrument defined by a leadscrew and a lead nut;

FIG. 32A depicts a side cross-sectional view of the handle assembly of the instrument of FIG. 29A, taken along line 32A-32A in FIG. 29A, with the lead nut in a distal position;

FIG. 32B depicts a side cross-sectional view of the handle assembly of the instrument of FIG. 29A, with the lead nut in a proximal position;

FIG. 33 depicts a side cross-sectional view of another example of a handle assembly for use with the instrument of FIG. 29A, with a deflection control knob that is integrally formed together with a leadscrew as a unitary piece, and with a suction valve having a dedicated suction inlet port;

FIG. 34A depicts a schematic view of an example of an irrigation fluid dispensing assembly for use with any of the instruments of FIGS. 2A and 29A, with an irrigation fluid bladder in an expanded state;

FIG. 34B depicts a schematic view of the irrigation fluid dispensing assembly of FIG. 34A, with the irrigation fluid bladder in a contracted state;

FIG. 35 depicts a front elevation view of another example of a distal endoscope cap for use with the instrument of FIG. 2A and having two cameras, a single illuminating element, and a single navigation sensor; and

FIG. 36 depicts a top cross-sectional view of another example of a distal endoscope cap for use with the instrument of FIG. 2A and having two cameras, a single illuminating element, a single navigation sensor, and a single optical sensor.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

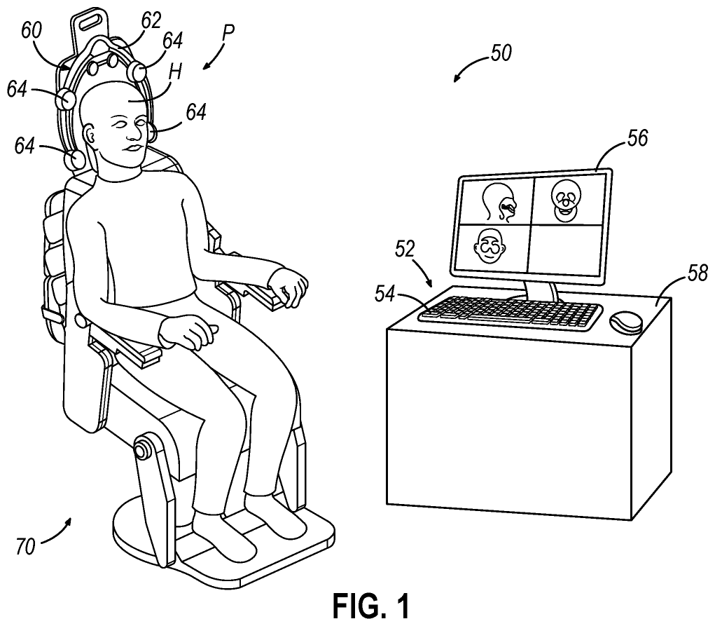

When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). FIG. 1 shows an example of a IGS navigation system (50) enabling an ENT procedure to be performed using image guidance. In addition to or in lieu of having the components and operability described herein IGS navigation system (50) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein.

IGS navigation system (50) of the present example comprises a field generator assembly (60), which comprises set of magnetic field generators (64) that are integrated into a horseshoe-shaped frame (62). Field generators (64) are operable to generate alternating magnetic fields of different frequencies around the head (H) of the patient (P). An instrument, such as any of the ablation instruments described below, may be inserted into the head (H) of the patient (P). Such an instrument may be a standalone device or may be positioned on an end effector. In the present example, frame (62) is mounted to a chair (70), with the patient (P) being seated in the chair (70) such that frame (62) is located adjacent to the head (H) of the patient (P). By way of example only, chair (70) and/or field generator assembly (60) may be configured and operable in accordance with at least some of the teachings of U.S. Pat. No. 10,561,370, entitled “Apparatus to Secure Field Generating Device to Chair,” issued Feb. 18, 2020, the disclosure of which is incorporated by reference herein.

IGS navigation system (50) of the present example further comprises a processor (52), which controls field generators (64) and other elements of IGS navigation system (50). For instance, processor (52) is operable to drive field generators (64) to generate alternating electromagnetic fields; and process signals from the instrument to determine the location of a navigation sensor in the instrument within the head (H) of the patient (P). Processor (52) comprises a processing unit (e.g., a set of electronic circuits arranged to evaluate and execute software instructions using combinational logic circuitry or other similar circuitry) communicating with one or more memories. Processor (52) of the present example is mounted in a console (58), which comprises operating controls (54) that include a keypad and/or a pointing device such as a mouse or trackball. A physician uses operating controls (54) to interact with processor (52) while performing the surgical procedure.

While not shown, the instrument may include a navigation sensor that is responsive to positioning within the alternating magnetic fields generated by field generators (64). A coupling unit (not shown) may be secured to the proximal end of the instrument and may be configured to provide communication of data and other signals between console (58) and the instrument. The coupling unit may provide wired or wireless communication of data and other signals.

In some versions, the navigation sensor of the instrument may comprise at least one coil at or near the distal end of the instrument. When such a coil is positioned within an alternating electromagnetic field generated by field generators (64), the alternating magnetic field may generate electrical current in the coil, and this electrical current may be communicated along the electrical conduit(s) in the instrument and further to processor (52) via the coupling unit. This phenomenon may enable IGS navigation system (50) to determine the location of the distal end of the instrument within a three-dimensional space (i.e., within the head (H) of the patient (P), etc.). To accomplish this, processor (52) executes an algorithm to calculate location coordinates of the distal end of the instrument from the position related signals of the coil(s) in the instrument.

Processor (52) uses software stored in a memory of processor (52) to calibrate and operate IGS navigation system (50). Such operation includes driving field generators (64), processing data from the instrument, processing data from operating controls (54), and driving display screen (56). In some implementations, operation may also include monitoring and enforcement of one or more safety features or functions of IGS navigation system (50). Processor (52) is further operable to provide video in real time via display screen (56), showing the position of the distal end of the instrument in relation to a video camera image of the patient's head (H), a CT scan image of the patient's head (H), and/or a computer-generated three-dimensional model of the anatomy within and adjacent to the patient's nasal cavity. Display screen (56) may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, display screen (56) may provide images in accordance with at least some of the teachings of U.S. Pat. No. 10,463,242, entitled “Guidewire Navigation for Sinuplasty,” issued Nov. 5, 2019, the disclosure of which is incorporated by reference herein. In the event that the operator is also using an endoscope, the endoscopic image may also be provided on display screen (56).

The images provided through display screen (56) may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

### II. Examples of a Distal Endoscope Cap for ENT Instruments

In some instances, it may be desirable to provide a distal endoscope cap configured for selective attachment to a distal end of an ENT instrument for retrofitting or otherwise modifying the ENT instrument to provide the ENT instrument with visualization and/or navigation capabilities. For example, retrofitting the ENT instrument with such a distal endoscope cap may free the shaft of ENT instrument from competing with a separate endoscope shaft for space within small, tortuous anatomical passageways within the patient's head (H). In addition, or alternatively, such a distal endoscope cap may provide visualization of a field of view that is in-line with the operative field at which the ENT instrument is performing treatment, rather than the field of view being offset from the operative field as may be the case when a separate endoscope is used. Each of the examples of distal endoscope caps (110, 210, 310, 410, 510, 610, 710, 1410, 1510) described below may function in such a manner. While the examples provided below are discussed in the context of a particular ENT instrument (100), distal endoscope caps (110, 210, 310, 410, 510, 610, 710, 1410, 1510) may be used to provide navigation capabilities to any other suitable ENT instruments. Other suitable ways in which distal endoscope caps (110, 210, 310, 410, 510, 610, 710, 1410, 1510) may be used will be apparent to those skilled in the art in view of the teachings herein.

### A. Example of an Instrument with Distal Endoscope Cap

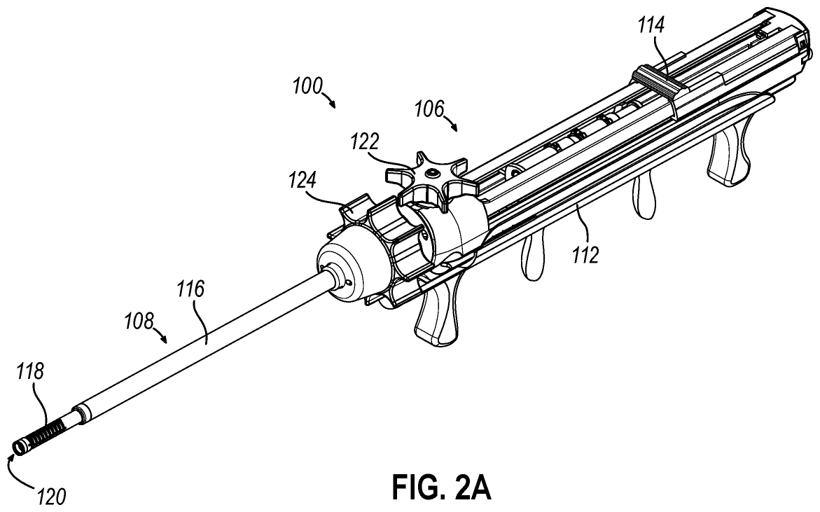

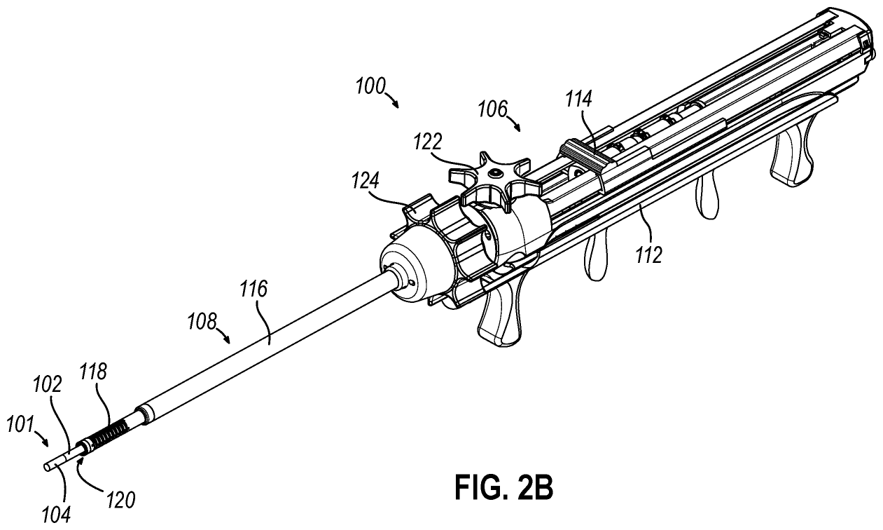

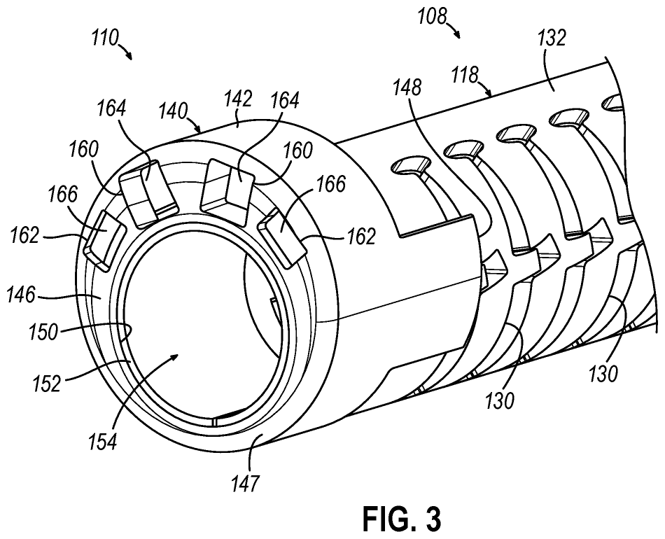

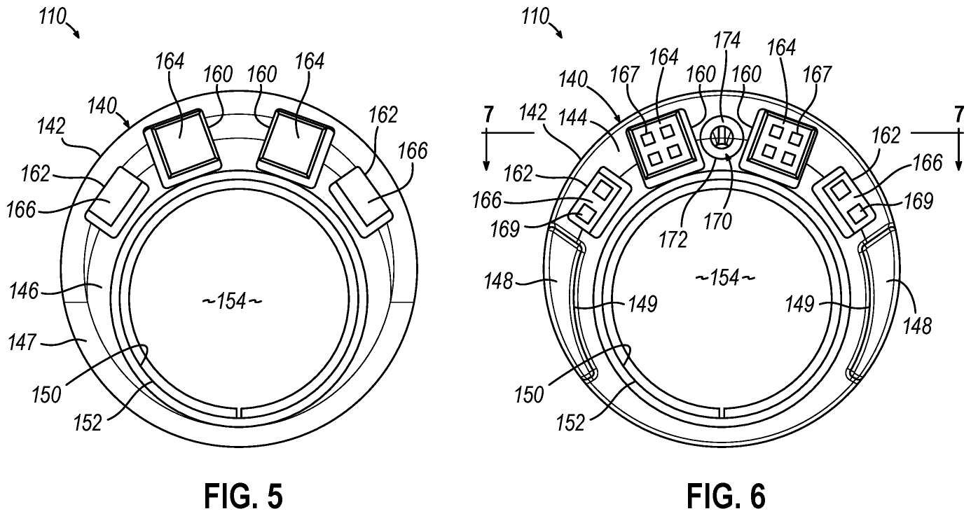

FIGS. 2A-7 show an example of an instrument (100) that may be used to guide a working element (101) (FIG. 2B) into an anatomical passageway, and to which an example of a distal endoscope cap (110) (FIGS. 3-7) may be either removably or permanently attached. As shown in FIG. 2B, working element (101) includes a shaft (102) and an end effector (104). In some versions, working element (101) may include a dilation catheter. In this regard, end effector (104) may have one or more balloons or other dilators, such that instrument (100) may be used to guide end effector (104) of working element (101) into an anatomical passageway to thereby dilate the anatomical passageway. For instance, instrument (100) and working element (101) may be used for dilation of ostia of paranasal sinuses (e.g., to treat sinusitis), dilation of the larynx, dilation of the Eustachian tube, dilation of other passageways within the ear, nose, or throat, etc. In addition, or alternatively, working element (101) may include an electrical energy (e.g., RF energy and/or pulsed field DC energy, etc.) delivery catheter. In this regard, end effector (104) may have one or more electrodes, such that instrument (100) may be used to guide end effector (104) of working element (101) into an anatomical passageway to deliver electrical energy to tissue in or near the anatomical passageway. For instance, instrument (100) and working element (101) may be used to ablate a nerve (e.g., a posterior nasal neve); ablate a turbinate; or ablate, electroporate (e.g., to promote absorption of therapeutic agents, etc.), or apply resistive heating to any other kind of anatomical structure in the head of a patient. It will be appreciated that working element (101) may include any other suitable type of ENT treatment device.

Instrument (100) of this example includes a handle assembly (106) and a shaft assembly (108). Instrument (100) may be coupled with an inflation fluid source (not shown), which may be operable to selectively supply an inflation fluid to a balloon (not shown) of end effector (104), for inflating the balloon to thereby dilate the anatomical passageway. In addition, or alternatively, instrument (100) may be coupled with an ablation energy generator (not shown), which may be operable to generate ablation energy for delivery to tissue via electrodes (not shown) of end effector (104)) to thereby ablate, electroporate, or apply resistive heating to the tissue. Energy produced by the ablation energy generator may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Handle assembly (106) of this example includes a body (112) and at least one slider (114). Body (112) is sized and configured to be grasped and operated by a single hand of an operator, such as via a power grip, a pencil grip, or any other suitable kind of grip. Slider (114) is operable to translate longitudinally relative to body (112). Slider (114) is coupled with working element (101) and is thus operable to translate working device (101) longitudinally between a proximally retracted position (FIG. 2A) and a distally extended position (FIG. 2B). In some versions, another slider (not shown) may be operable to translate a guidewire (not shown) longitudinally for directing working device (101) therealong.

Shaft assembly (108) of the present example includes a rigid portion (116), a flexible portion (118) distal to rigid portion (116), and an open distal end (120). A pull-wire (not shown) is coupled with flexible portion (118) and with a deflection control knob (122) of handle assembly (106). Deflection control knob (122) is rotatable relative to body (112), about an axis that is perpendicular to the longitudinal axis of shaft assembly (108), to selectively retract the pull-wire proximally. As the pull-wire is retracted proximally, flexible portion (118) bends and thereby deflects distal end (120) laterally away from the longitudinal axis of rigid portion (116). Deflection control knob (122), the pull-wire, and flexible portion (118) thus cooperate to impart steerability to shaft assembly (108). By way of example only, such steerability of shaft assembly (108) may be provided in accordance with at least some of the teachings of U.S. Pat. Pub. No. 2021/0361912, entitled “Shaft Deflection Control Assembly for ENT Guide Instrument,” published Nov. 25, 2021, the disclosure of which is incorporated by reference herein, in its entirety. Other versions may provide some other kind of user input feature to drive steering of flexible portion (118), instead of deflection control knob (122). In some alternative versions, deflection control knob (122) is omitted, and flexible portion (118) is malleable. In still other versions, the entire length of shaft assembly (108) is rigid.

Shaft assembly (108) is also rotatable relative to handle assembly (106), about the longitudinal axis of rigid portion (116). Such rotation may be driven via rotation control knob (124), which is rotatably coupled with body (112) of handle assembly (106). Alternatively, shaft assembly (108) may be rotated via some other form of user input; or may be non-rotatable relative to handle assembly (106). It should also be understood that the example of handle assembly (106) described herein is merely an illustrative example. Shaft assembly (108) may instead be coupled with any other suitable kind of handle assembly or other supporting body.

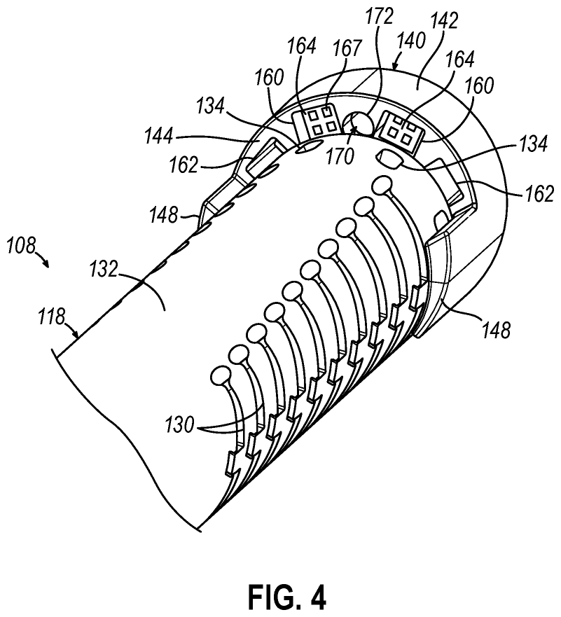

As best shown in FIGS. 3-4, flexible portion (also referred to as a flexible guide shaft or a deflectable guide shaft) (118) of shaft assembly (108) includes a linear array of articulating ribs (130) connected to each other by a resilient spine (132) for accommodating articulation of articulating ribs (130) relative to rigid portion (116) via the pull-wire described above. To that end, flexible portion (118) also includes a pair of pull-wire coupling holes (134) near open distal end (120) and generally angularly aligned with resilient spine (132) about the longitudinal axis of flexible portion (118) for coupling with a distal end of the pull-wire. A working lumen (not shown) extends longitudinally from an open proximal end (not shown) of shaft assembly (108) all the way to open distal end (120) and is configured to slidably receive working element (101), such that shaft assembly (108) may receive working element (101) at the open proximal end, and such that shaft assembly (108) may guide working element (101) out through open distal end (120). In some versions, the working lumen may have a diameter of about 2.9 mm. Flexible portion (118) of shaft assembly (108) may be formed of a metallic material, such as stainless steel and/or nitinol. In addition, or alternatively, flexible portion (118) may be configured and operable in accordance with any one or more of the teachings of U.S. Pat. No. 11,376,401, entitled “Deflectable Guide for Medical Instrument,” issued Jul. 5, 2022, the disclosure of which is incorporated by reference herein.

With continuing reference to FIGS. 3-4, distal endoscope cap (110) is attachable to open distal end (120) of shaft assembly (108) and is operable to provide visualization, navigation, and irrigation capabilities to shaft assembly (108) while allowing shaft assembly (108) to continue to be used to guide working element (101) through open distal end (120).

In this regard, distal endoscope cap (110) of the present example includes a body (140) having a generally cylindrical hub (142) extending between proximal and distal surfaces (144, 146). Distal endoscope cap (110) of the present example includes a fillet (147) between distal surface (146) and a generally cylindrical outer surface of hub (142) to inhibit trauma to tissue encountered by distal endoscope cap (110), such that distal endoscope cap (110) may be considered atraumatic. In the example shown, body (140) also has a pair of laterally-opposed coupling wings (148) extending proximally from proximal surface (144) of hub (142). Body (140) may be constructed of any suitable translucent or opaque material, such as a polymeric material (e.g., plastic) or a metallic material. In the example shown, hub (142) and wings (148) are integrally formed together with each other as a unitary (e.g., monolithic) piece to define body (140). In this regard, body (140) may be manufactured via 3D printing, injection molding, investment casting, machining, and/or any other suitable manufacturing techniques. In some versions, distal endoscope cap (110) may have a maximum outer cross-dimension, such as a maximum outer diameter (e.g., defined by the cylindrical outer surface of hub (142)), less than about 4.3 mm, such as less than about 4.2 mm. In this manner, the maximum outer cross-dimension of distal endoscope cap (110) may be only slightly greater than that of flexible portion (118) of shaft assembly (108) (which may itself be between about 3.6 mm and about 3.7 mm) to avoid interfering with the ability of flexible portion (118) to fit within anatomical passageways when distal endoscope cap (110) is attached thereto.

As best shown in FIG. 6, each wing (148) of distal endoscope cap (110) includes a laterally inwardly-facing gripping surface (149) configured to frictionally engage a generally cylindrical outer surface of flexible portion (118) of shaft assembly (108) near open distal end (120) for removably attaching distal endoscope cap (110) to open distal end (120). In the example shown, gripping surfaces (149) are spaced apart from each other by a distance substantially equal to or slightly less than a diameter of the cylindrical outer surface of flexible portion (118), and each gripping surface (149) is curved to complement the cylindrical outer surface of flexible portion (118) for enhancing the frictional engagement between each gripping surface (149) and the cylindrical outer surface of flexible portion (118). In some versions, each wing (148) may be configured to flex slightly laterally outwardly during insertion of flexible portion (118) between gripping surfaces (149). While gripping surfaces (149) of wings (148) are shown frictionally engaging the cylindrical outer surface of flexible portion (118), it will be appreciated that distal endoscope cap (110) may be either removably or permanently attached to open distal end (120) in any suitable manner, such as via adhesive, thermal bonding, welding, snap fit, or any other attachment techniques.

Distal endoscope cap (110) of the present example also includes a generally cylindrical bore (150) extending longitudinally between proximal and distal surfaces (144, 146) of hub (142) and configured to be axially aligned with the working lumen of shaft assembly (108) when distal endoscope cap (110) is attached to open distal end (120), such that working element (101) may pass through bore (150) as working element (101) is guided through distal open end (120). In some versions, bore (150) may have an inner cross-dimension (e.g., diameter) of between about 2.5 mm and about 2.9 mm. Alternatively, bore (150) may have any other suitable inner cross-dimension.

As used herein, the term “axially aligned” should not be read as necessarily requiring that the central axis of bore (150) must be coaxial with the central axis of the working lumen of shaft assembly (108). Instead, the term “axially aligned” should be read as including arrangements where the central axis of the working lumen of shaft assembly (108) passes through bore (150), with the central axis of the working lumen of shaft assembly (108) being laterally offset from the central axis of bore (150). “Axially aligned” thus includes any arrangements where a working element (101) that is advanced along the working lumen of shaft assembly (108) may ultimately pass through bore (150). Of course, some versions of “axially aligned” arrangements may include arrangements where the central axis of bore (150) is coaxial with the central axis of the working lumen of shaft assembly (108).

In the example shown, distal endoscope cap (110) further includes a navigation sensor assembly (152) received within bore (150) and configured to provide navigation capabilities to shaft assembly (108). Navigation sensor assembly (152) includes at least one electromagnetic coil (not shown) operable to generate signals indicative of the position of the respective coil and thereby indicative of distal endoscope cap (110) and/or a portion of shaft assembly (108) (e.g., flexible portion (118)) in three-dimensional space when positioned within an alternating electromagnetic field generated by field generators (64). The position data generated by such position related signals may be processed by processor (52) for providing a visual indication to the operator to show the operator where shaft assembly (108) of instrument (100) is located within the patient (P) in real time. Such a visual indication may be provided as an overlay on one or more preoperatively obtained images (e.g., CT scans) of the patient's anatomy. Navigation sensor assembly (152) may be configured as a single-axis sensor (SAS) (e.g., having a single electromagnetic coil wound about a single axis), as a dual-axis sensor (DAS) (e.g., having two electromagnetic coils wound about respective axes), or as a triple-axis sensor (TAS) (e.g., having three electromagnetic coils wound about respective axes). In addition, or alternatively, navigation sensor assembly (152) may be configured as a flexible printed circuit board (PCB). By way of example only, navigation sensor assembly (152) may be configured and operable in accordance with at least some of the teachings of U.S. Pat. Pub. No. 2022/0257093, entitled “Flexible Sensor Assembly for ENT Instrument,” published Aug. 18, 2022, the disclosure of which is incorporated by reference herein, in its entirety.

Navigation sensor assembly (152) is suitably sized to fit within bore (150) while still permitting space for a working channel (154) to extend along bore (150) (e.g., radially inwardly relative to navigation sensor assembly (152)), thereby permitting additional instrumentation (e.g., working element (101)), suction, fluids, etc. to pass between working channel (154) and the working lumen of shaft assembly (108). In this regard, navigation sensor assembly (152) may be disposed along a generally cylindrical inner surface of bore (150) and may have a generally curved configuration such that navigation sensor assembly (152) is curved about the longitudinal axis of flexible portion (118) of shaft assembly (108) with a radius of curvature corresponding to that of the cylindrical inner surface to thereby conform to an inner circumference of bore (150). In the example shown, navigation sensor assembly (152) has a length greater than that of bore (150), such that a distal portion of navigation sensor assembly (152) is received within the working lumen of shaft assembly (108). In other versions, navigation sensor assembly (152) may have a length substantially equal to or less than that of bore (150), such that navigation sensor assembly (152) may be entirely housed within bore (150).

Alternatively, navigation sensor assembly (152) may be disposed along a generally cylindrical outer surface of hub (142) of distal endoscope cap (110), such as along either a top or bottom portion of the cylindrical outer surface of hub (142), and may have a generally curved configuration such that navigation sensor assembly (152) is curved about the longitudinal axis of hub (142) of distal endoscope cap (110) with a radius of curvature corresponding to that of the cylindrical outer surface to thereby conform to an outer circumference of hub (142). In other versions, navigation sensor assembly (152) may be disposed between flexible portion (118) of shaft assembly (108) and distal endoscope cap (110), or within an at least partially annular slot (not shown) extending at least partially through hub (142) of distal endoscope cap (110). In such cases, working channel (154) may be defined by the generally cylindrical inner surface of bore (150). In some versions, body (140) may be configured to electrically isolate the electromagnetic coil(s) of navigation sensor assembly (152) from flexible portion (118) of shaft assembly (108) to inhibit electrical shorting between the electromagnetic coil(s) and flexible portion (118) and/or to inhibit generation of electromagnetic interference or “noise.” For example, body (140) may be constructed of an electrically non-conductive (e.g., insulative) material. In some other versions, one or more navigation sensor assemblies (152) may be disposed on one or both wings (148).

In any event, navigation sensor assembly (152) may permit space for working channel (154) to extend along bore (150) as described above, such that navigation sensor assembly (152) may continuously communicate position related signals to processor (52) during advancement of working element (101) distally through working channel (154) and/or while working element (101) remains positioned within working channel (154), such as during inflation of a balloon of end effector (104) to dilate an anatomical passageway and/or during delivery of electrical energy to tissue via electrodes of end effector (104). In other words, navigation of flexible portion (118) may be performed concurrently with dilation of an anatomical passageway and/or concurrently with delivery of electrical energy to tissue, without interfering with each other. To that end, working channel (154) may have an inner cross-dimension (e.g., diameter) of at least about 2.5 mm, such that working element (101) may have a size on the French catheter scale of between about 0 Fr and about 7 Fr, for example. In some versions, working channel (154) is in fluid communication with a source of suction (not shown), such as via the working lumen of shaft assembly (108), for reasons described below.

Distal endoscope cap (110) of the present example also includes an arch-shaped array of generally rectangular bores (160, 162) each extending longitudinally between proximal and distal surfaces (144, 146) of hub (142) and disposed about (e.g., above) bore (150). More particularly, distal endoscope cap (110) includes an inner pair of laterally-opposed bores (160) and an outer pair of laterally-opposed bores (162).

In the example shown, distal endoscope cap (110) further includes a pair of imaging devices (also referred to as image sensors) in the form of cameras (164) received within respective inner bores (160) and a pair of illuminating elements (166) received within respective outer bores (162). In some versions, bores (160, 162) may each be sized and shaped to provide a snap fit between the bore (160, 162) and the corresponding camera (164) or illuminating element (166). It will be appreciated that cameras (164) and illuminating elements (166) may be fixedly retained within the respective bores (160, 162) in any other suitable manner.

In any event, cameras (164) and illuminating elements (166) are configured to cooperate with each other to provide visualization capabilities to shaft assembly (108). Cameras (164) are spaced apart from each other within the respective inner bores (160) to provide stereoscopic visualization of objects within the collective field of view of cameras (164). For example, cameras (164) may capture real-time 3D images of a patient's anatomy and thus enable acquisition of depth-of-field data and/or integration of artificial intelligence programs. In some cases, the images captured by cameras (164) may be superimposed on preoperatively obtained images to provide one or more augmented reality views. In some cases, such an augmented reality view may be updated in real time to reflect anatomical modifications made by working element (101) or other components of instrument (100) (e.g., tissue ablation, dilation, etc.). In addition, or alternatively, cameras (164) may be used to intraoperatively optically track the locations of other components within the patient's anatomy, such as working element (101). Such components may include one or more fiducial markers (not shown) that may be optically recognized via cameras (164). Cameras (164) may thereby provide internal registration of such components. By way of example only, cameras (164) may be configured and operable in accordance with at least some of the teachings of U.S. Pat. No. 10,955,657, entitled “Endoscope with Dual Image Sensors,” issued Mar. 23, 2021, the disclosure of which is incorporated by reference herein; U.S. Pat. Pub. No. 2020/0196851, entitled “3D Scanning of Nasal Tract with Deflectable Endoscope,” published Jun. 25, 2020, the disclosure of which is incorporated by reference herein; and/or U.S. Pat. No. 11,457,981, entitled “Computerized Tomography (CT) Image Correction Using Position and Direction (P&D) Tracking Assisted Optical Visualization,” issued Oct. 4, 2022, the disclosure of which is incorporated by reference herein. In some cases, the centers of cameras (164) may be spaced apart from each other by a distance of between about 1 mm and about 2 mm. In the example shown, cameras (164) are oriented relative to each other along a curve defined by the arch-shaped array of generally rectangular bores (160, 162) and are each at least slightly recessed proximally relative to distal surface (146) of hub (142), as described in greater detail below.

As best shown in FIG. 6, each camera (164) has a plurality of leads (167) on a proximal end thereof configured to be operatively coupled to processor (52) and/or electrically coupled to a power source (not shown) via respective traces or wires (not shown) extending proximally through the respective bore (160) and along shaft assembly (108) to the coupling unit, for example. Each camera (164) of the present version is fixedly retained within the respective inner bore (160). In some cases, one or more alignment protrusions may be provided in each inner bore (160) for abutting the respective camera (164) to promote positioning of each camera (164) at a predetermined longitudinal position.

In some versions, one or both cameras (164) may be configured to extend distally from the respective inner bore(s) (160) beyond distal surface (146) of hub (142) (e.g., for accessing and visualizing areas of the patient's anatomy that may not be readily accessible by the entire distal endoscope cap (110)) and retract proximally into the respective inner bore(s) (160). For example, each camera (164) may be extendable and retractable independently of the other camera (164). In any event, each camera (164) may be at least slightly recessed proximally relative to distal surface (146) of hub (142) as shown to inhibit snagging of cameras (164) on tissue during insertion into the patient's head (H). Such recessed placement of each camera (164) relative to distal surface (146) of hub (142) may also assist with allowing the distal end of each camera (164) to be flushed with liquid, as described in greater detail below.

Illuminating elements (166) are configured and operable to illuminate the field of view of cameras (164). Each illuminating element (166) is positioned outboard relative to the adjacent camera (164). It will be appreciated that illuminating elements (166) may provide an increased maximum brightness, at least by comparison to a single illuminating element (166). In some versions, each illuminating element (166) may be operated (e.g., via processor (52)) independently of the other illuminating element (166) for independently adjusting the light intensity of each illuminating element (166), such as to reflect light off of a patient's tissues and provide illumination at two different discrete depths of field or anatomical structures. In some versions, the light intensity of each illuminating element (166) may be adjusted via processor (52) using an artificial intelligence program. While two illuminating elements (166) are used in the present example, other versions may employ just one illuminating element (166) or more than two illuminating elements (166). In the present example, illuminating elements (166) include LEDs.

As best shown in FIG. 6, each illuminating element (166) has a pair of leads (169) on a proximal end thereof configured to be electrically coupled to a power source (not shown) via respective traces or wires (not shown) extending proximally through the respective bore (162) and along shaft assembly (108) to the coupling unit, for example. In some other versions, one or both illuminating elements (166) may include fiber optic components. For instance, each illuminating element (166) may include a lens that is optically coupled with one or more respective optical fibers or optical fiber bundles. Such optical fibers or optical fiber bundles may extend along shaft assembly (108) and be optically coupled with a source of light that is either integrated into handle assembly (106) (or some other body from which shaft assembly (108) extends) or otherwise provided. In addition, or alternatively, one or both illuminating elements (166) may include a laser.

While bores (160, 162) are shown for receiving cameras (164) and illuminating elements (166), it will be appreciated that cameras (164) and illuminating elements (166) may be coupled to body (140) in any other suitable manner. For example, distal endoscope cap (110) may include an arch-shaped array of recesses (not shown) extending radially outwardly from bore (150) and longitudinally between proximal and distal surfaces (144, 146) of hub (142) in place of bores (160, 162). Such recesses may each be configured to receive a corresponding camera (164) or illuminating element (166). In some versions, the recesses may each be sized and shaped to provide a snap fit between the recess and the corresponding camera (164) or illuminating element (166). It will be appreciated that such recesses may enable each camera (164) and illuminating element (166) to be initially inserted together with the respective wires (not shown) longitudinally through bore (150), then pressed radially outwardly into the corresponding recess. In this regard, navigation sensor assembly (152) may be inserted into bore (150) after cameras (164) and illuminating elements (166) have been positioned within the respective recesses to cover the radially inner ends of the recesses and thereby assist with retaining cameras (164) and illuminating elements (166) together with the respective wires within the corresponding recesses.

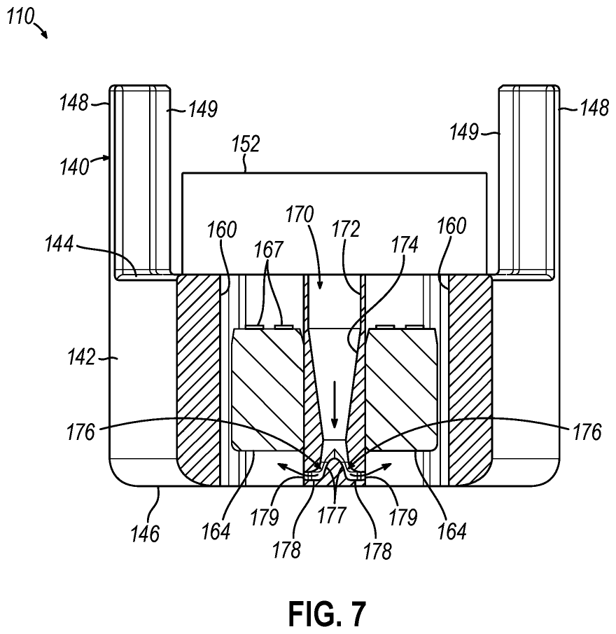

As best shown in FIG. 7, distal endoscope cap (110) of the present example also includes an irrigation lumen (170) extending distally from proximal surface (144) toward distal surface (146) of hub (142) and disposed above bore (150) between inner bores (160). More particularly, irrigation lumen (170) includes a proximal inlet port (172) extending distally from proximal surface (144), a medial fluid constriction passageway (174) downstream of inlet port (172), and a pair of laterally-opposed distal fluid diversion passageways (176) downstream of fluid constriction passageway (174) and in fluid communication with respective inner bores (160) slightly proximally of distal surface (146). Inlet port (172) may be configured to receive a distal end and/or a distal fluid coupling of an irrigation fluid conduit, such as a tube (not shown), to provide a fluid-tight connection between irrigation lumen (170) and a lumen of the irrigation fluid conduit, such that irrigation lumen (170) may be in fluid communication with a source of irrigation fluid (e.g., saline or any other suitable liquid) via the irrigation fluid conduit. Fluid constriction passageway (174) of the present example tapers radially inwardly toward a longitudinal axis thereof in a distal direction such that fluid constriction passageway (174) may be configured to increase the fluid pressure of liquid traveling distally therethrough from inlet port (172) and to thereby increase the fluid flow rate of such liquid.

Fluid diversion passageways (176) of the present example each include an upstream portion (177) extending slightly obliquely relative to the longitudinal axis of fluid constriction passageway (174) to diverge the liquid from fluid constriction passageway (174) into two separate streams. Fluid diversion passageways (176) of the present example each further include a downstream portion (178) extending generally laterally outwardly relative to the longitudinal axis of fluid constriction passageway (174) to direct the corresponding stream of liquid generally laterally outwardly into the respective inner bore (160) through a corresponding fluid outlet slot (179). It will be appreciated that the streams of liquid expelled from fluid outlet slots (179) may also be directed at least partially proximally. For example, the fluid flow rate of each stream traveling through fluid diversion passageways (176) may be sufficiently great to cause each stream to be deflected at least slightly proximally by respective distal surfaces of downstream portions (178) upon impacting such distal surfaces. In addition, or alternatively, each downstream portion (178) may be oriented slightly obliquely relative to the lateral direction to direct the streams of liquid expelled from fluid outlet slots (179) both laterally outwardly and proximally.

Fluid outlet slots (179) of the present example each extend along a laterally-inner surface of the corresponding inner bore (160) at or near a distal end thereof, such that fluid outlet slots (179) are laterally inward of and at least slightly distal relative to the respective cameras (164). In this manner, fluid outlet slots (179) may each direct a respective stream of the liquid from fluid constriction passageway (174) laterally outwardly and/or proximally toward a distal end (e.g., lens) of the respective camera (164). Thus, when liquid is conveyed along irrigation lumen (170), and such liquid exits fluid outlet slots (179), the expelled liquid may be directed toward the distal end of the respective camera (164) and thereby flush debris (e.g., blood, tissue, mucus, etc.) off of and/or away from the respective camera (164). By flushing the distal ends of cameras (164) with liquid, irrigation lumen (170) may be used to keep the distal ends of cameras (164) clear of debris and thereby maintain appropriate visualization via cameras (164) without requiring withdrawal of instrument (100) from the patient's head (H) for manual cleaning of cameras (164). As described above, working channel (154) may be in fluid communication with a source of suction, and may thus be used to draw excess liquids (e.g., liquid expelled via fluid outlet slots (179), etc.) and debris away from cameras (164). In such cases, working channel (154) may define a suction lumen of distal endoscope cap (110). In other versions, distal endoscope cap (110) may include one or more suction lumens separate from working channel (154).

While fluid outlet slots (179) are shown extending along laterally-inner surfaces of inner bores (160) for directing separate streams of liquid toward the distal ends of cameras (164), it will be appreciated that one or more streams of liquid may be directed toward the distal ends of cameras (164) in any other suitable manner. For example, irrigation lumen (170) may include an array of fluid outlet bores (not shown) in place of each fluid outlet slot (179). Such arrays may each include a plurality of rows and columns of fluid outlet bores, and fluid outlet bores of each array may be oriented at various different angles so that the fluid outlet bores of each array may collectively direct liquid toward substantially the entire distal end of the respective camera (164) in a sprinkler-type arrangement. In other versions, irrigation lumen (170) may be disposed above inner bores (160), and may include one or more arcuate fluid outlet slots (not shown) above and at least slightly distal relative to both cameras (164) for directing one or more streams of liquid downwardly toward the distal ends of cameras (164) and/or illuminating elements (166) in a water curtain-type arrangement. In some versions, a distal overhang (not shown) may be in-line with and distal of the one or more arcuate fluid outlet slots for assisting with directing the stream(s) of liquid downwardly.

In the example shown, irrigation lumen (170) of distal endoscope cap (110) is generally angularly aligned with resilient spine (132) of flexible portion (118) of shaft assembly (108) about the longitudinal axis of flexible portion (118) for facilitating routing of the irrigation fluid conduit to irrigation lumen (170) along resilient spine (132). The flanking of irrigation lumen (170) by inner bores (160) and the flanking of inner bores (160) by outer bores (162) may further facilitate routing of wires to cameras (164) and illuminating elements (166) along resilient spine (132). It will be appreciated that routing the irrigation fluid conduit and/or wires along resilient spine (132) may allow the irrigation fluid conduit and/or wires to avoid interfering with the ability of flexible portion (118) to bend. In some versions, the irrigation fluid conduit and/or wires may be secured to flexible portion (118) via a heat shrink sleeve or any other suitable attachment mechanism, such as any of the routing devices described below.

In some versions, body (140) may be electrically conductive and configured to deliver electrical energy to tissue. For example, body (140) may be coupled with a corresponding one or more wires routed along flexible portion (118) that electrically couple body (140) with an ablation energy generator, such as an RF generator. Body (140) may thereby serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between body (140) and flexible portion (118), such that body (140) may be electrically energized without also energizing flexible portion (118) or other portions of shaft assembly (108).

In some other versions, distal endoscope cap (110) may include one or more electrically conductive elements secured to body (140) and configured to deliver electrical energy to tissue. For example, a pair of arcuate conductive elements may be angularly spaced apart from each other on distal surface (146) of hub (142). Such conductive elements may each include any one or more of a conductive wire, plate, film, and/or coating, and may be formed of any suitable material or combination of materials including but not limited to metallic conductive materials such as copper, gold, steel, aluminum, silver, nitinol, etc. and/or non-metallic conductive materials such as conducting polymers, silicides, graphite, etc. Such conductive elements may be secured to body (140) in any suitable fashion, including but not limited to being secured via an adhesive, via vapor deposition, or otherwise, and may be coupled with a corresponding one or more wires routed along flexible portion (118) that electrically couple such conductive elements with an ablation energy generator, such as an RF generator. The pair of conductive elements may thereby be operable to apply bipolar RF energy to tissue, with one conductive element serving as an active electrode and the other conductive element serving as a return electrode to ablate, electroporate, and/or cauterize the tissue, for example. In some versions, such conductive elements are configured and operable in accordance with at least some of the teachings of U.S. Pat. Pub. No. 2022/0110513, entitled “ENT Instrument with Deformable Guide having Translatable Imaging Feature,” published Apr. 14, 2022, the disclosure of which is incorporated by reference herein, in its entirety.

While instrument (100) has been described for dilating an anatomical passageway and/or for delivering electrical energy to tissue within the ear, nose, or throat of a patient, it will be appreciated that instrument (100) may be adapted to perform other surgical functions including, for example, diagnostic procedures, electrophysiology mapping, electrophysiology directed catheter guided surgery, and/or cardiac ablation procedures, such as via various other types of working elements (101).

### B. Distal Endoscope Cap Having Body with Two-Piece Construction

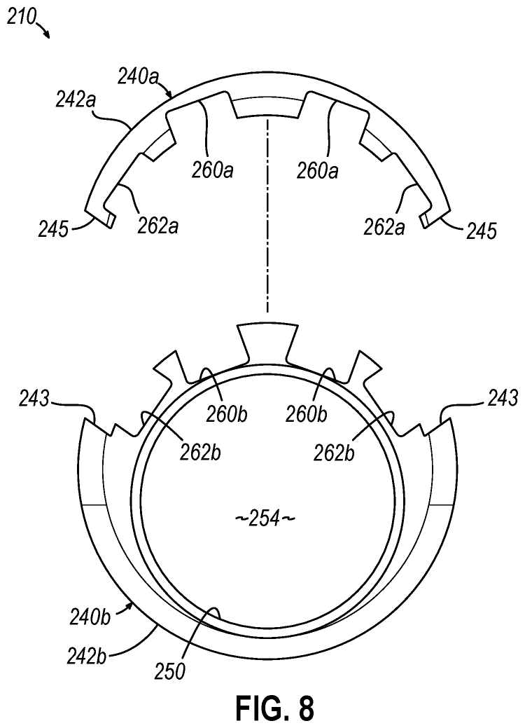

In some instances, it may be desirable to provide a distal endoscope cap having a body with a two-piece construction to facilitate or otherwise improve manufacturability of the distal endoscope cap, such as by enabling upper and lower portions of the irrigation lumen to be formed on upper and lower body portions, respectively, which may then be coupled to each other to define the body. In addition, or alternatively, such a two-piece construction of the body may enable upper and lower portions of the array of bores to be formed on the upper and lower body portions, respectively, so that the cameras and illuminating elements may be inserted into the lower portions of the respective bores, for example, and then covered by the upper body portion. FIGS. 8-10 show an example of a distal endoscope cap (210) having such functionalities, and which may be incorporated into instrument (100) in place of distal endoscope cap (110). Distal endoscope cap (210) may be similar to distal endoscope cap (110) described above except as otherwise described below. In this regard, distal endoscope cap (210) of the present example includes upper and lower body portions (240a, 240b) configured to be coupled to each other to collectively define a body similar to body (140).

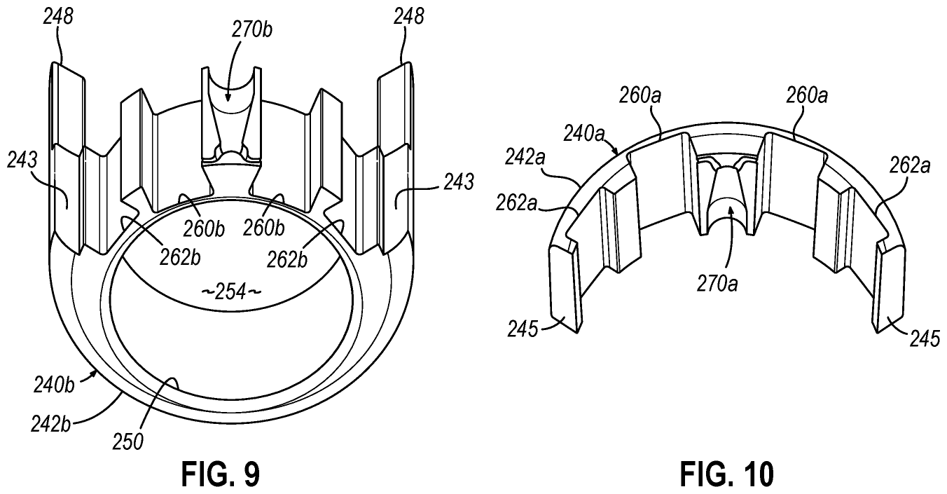

As shown, upper and lower body portions (240a, 240b) have upper and lower hub portions (242a, 242b), respectively, configured to collectively define a generally cylindrical hub similar to hub (142) when upper and lower body portions (240a, 240b) are coupled to each other. To that end, lower body portion (240b) has a pair of laterally-opposed notches (243) facing generally upwardly from lower hub portion (242b), and upper body portion (240a) has a pair of laterally-opposed edges (245) facing generally downwardly from upper hub portion (242a) and configured to mate with notches (243) for achieving proper alignment of body portions (240a, 240b). In some versions, one of notches (243) or edges (245) may include alignment pins and the other of notches (243) or edges (245) may include corresponding alignment bores for receiving the alignment pins. As best shown in FIG. 9, lower body portion (240b) also has a pair of laterally-opposed coupling wings (248) extending proximally from lower hub portion (242b). Body portions (240a, 240b) may each be constructed of any suitable translucent or opaque material, such as a polymeric material (e.g., plastic) or a metallic material. In the example shown, lower hub portion (242b) and wings (248) are integrally formed together with each other as a unitary (e.g., monolithic) piece to define lower body portion (240b). In this regard, body portions (240a, 240b) may each be manufactured via 3D printing, injection molding, investment casting, machining, and/or any other suitable manufacturing techniques. It will be appreciated that body portions (240a, 240b) may be either removably or permanently attached to each other in any suitable manner, such as via adhesive, thermal bonding, welding, snap fit, or any other attachment techniques.

Distal endoscope cap (210) of the present example also includes a generally cylindrical bore (250) extending longitudinally through lower hub portion (242b) and configured to be axially aligned with the working lumen of shaft assembly (108) when distal endoscope cap (210) is attached to open distal end (120), such that working element (101) may pass through bore (250) as working element (101) is guided through distal open end (120). While not shown, distal endoscope cap (210) may further include a navigation sensor assembly similar to navigation sensor assembly (152), which may be received within bore (250) or otherwise coupled to either body portion (240a, 240b) while permitting space for a working channel (254) to extend along bore (250) in a manner similar to that described above.

Distal endoscope cap (210) of the present example also includes an arch-shaped array of upper and lower generally rectangular bore portions (e.g., recesses) (260a, 260b, 262a, 262b) extending longitudinally along upper and lower hub portions (242a, 242b), respectively, configured to collectively define an array of generally rectangular bores similar to bores (160, 162) when upper and lower body portions (240a, 240b) are coupled to each other. More particularly, distal endoscope cap (210) includes an inner pair of upper bore portions (260a) extending radially outwardly from one or more lower, radially inner surfaces of upper body portion (240a), and an inner pair of lower bore portions (260b) extending radially inwardly from one or more upper, radially outer surfaces of lower body portion (240b), which are configured to collectively define an inner pair of laterally-opposed bores similar to bores (160) when upper and lower body portions (240a, 240b) are coupled to each other. Similarly, distal endoscope cap (210) includes an outer pair of upper bore portions (262a) extending radially outwardly from one or more lower, radially inner surfaces of upper body portion (240a), and an outer pair of lower bore portions (262b) extending radially inwardly from one or more upper, radially outer surfaces of lower body portion (240b), which are configured to collectively define an outer pair of laterally-opposed bores similar to bores (162) when upper and lower body portions (240a, 240b) are coupled to each other. While not shown, distal endoscope cap (210) may further include a pair of cameras similar to cameras (164), which may each be received within a respective inner bore defined by upper and lower inner bore portions (260a, 260b) in a manner similar to that described above, and/or a pair of illuminating elements similar to illuminating elements (166), which may each be received within a respective outer bore defined by upper and lower outer bore portions (262a, 262b) in a manner similar to that described above.

As best shown in FIGS. 9-10, distal endoscope cap (210) of the present example also includes upper and lower irrigation lumen portions (e.g., recesses) (270a, 270b) extending longitudinally along upper and lower hub portions (242a, 242b), respectively, configured to collectively define an irrigation lumen similar to irrigation lumen (170) when upper and lower body portions (240a, 240b) are coupled to each other. More particularly, distal endoscope cap (210) includes an upper irrigation lumen portion (270a) extending radially outwardly from one or more lower, radially inner surfaces of upper body portion (240a), and a lower irrigation lumen portion (270b) extending radially inwardly from one or more upper, radially outer surfaces of lower body portion (240b), which are configured to collectively define an irrigation lumen similar to irrigation lumen (270) when upper and lower body portions (240a, 240b) are coupled to each other.

In some versions, one of upper or lower body portions (240a, 240b) may be electrically conductive and configured to deliver electrical energy to tissue. For example, one of upper or lower body portions (240a, 240b) may be coupled with a corresponding one or more wires routed along flexible portion (118) that electrically couple the one of upper or lower body portions (240a, 240b) with an ablation energy generator, such as an RF generator. The one of upper or lower body portions (240a, 240b) may thereby serve as an electrode operable to cooperate with a ground pad (not shown) placed in contact with the patient's skin to apply monopolar RF energy to tissue to ablate, electroporate, and/or cauterize the tissue, for example. As another variation, upper body portion (240a) may be configured to apply RF energy at a first polarity, and lower body portion (240b) at a second polarity, such that upper and lower body portions (240a, 240b) may be able to cooperatively apply bipolar RF energy. In such versions, an electrically insulative material may be interposed between upper body portion (240a) and lower body portion (240b); and also between upper and lower body portions (240a, 240b) and flexible portion (118).

### C. Distal Endoscope Cap Having Body with Three-Piece Construction Including Plate Portion

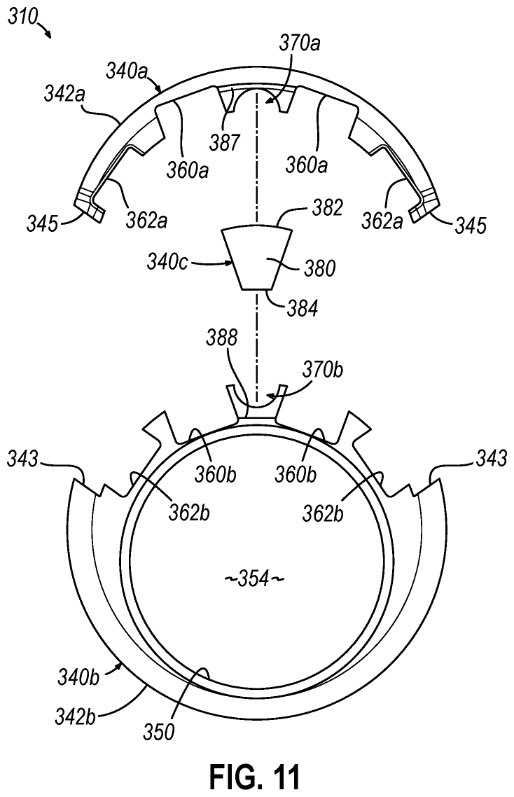

In some instances, it may be desirable to provide a distal endoscope cap having a body with a three-piece construction to facilitate or otherwise improve manufacturability of the distal endoscope cap, such as by enabling upper and lower portions of the irrigation lumen to be formed on upper and lower body portions, respectively, which may then be coupled to each other and to a fluid-diverting plate portion to define the body. In addition, or alternatively, such a three-piece construction of the body may enable upper and lower portions of the array of bores to be formed on the upper and lower body portions, respectively, so that the cameras and illuminating elements may be inserted into the lower portions of the respective bores, for example, and then covered by the upper body portion. FIGS. 11-14 show an example of a distal endoscope cap (310) having such functionalities, and which may be incorporated into instrument (100) in place of distal endoscope cap (110). Distal endoscope cap (310) may be similar to distal endoscope cap (110) described above except as otherwise described below. In this regard, distal endoscope cap (310) of the present example includes upper and lower body portions (340a, 340b) and a front plate portion (340c) configured to be coupled to each other to collectively define a body similar to body (140).

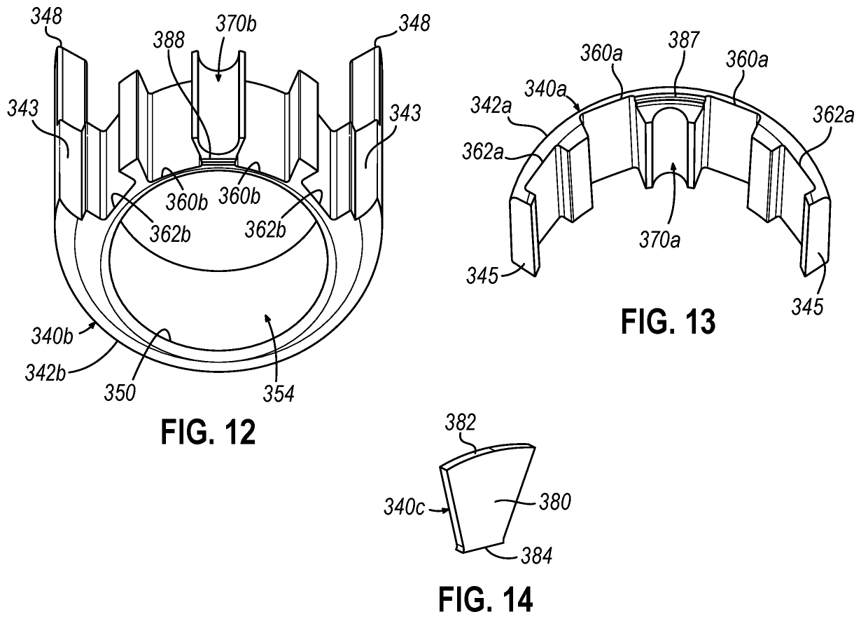

As shown, upper and lower body portions (340a, 340b) have upper and lower hub portions (342a, 342b), respectively, configured to collectively define a generally cylindrical hub similar to hub (142) when upper and lower body portions (340a, 340b) are coupled to each other. To that end, lower body portion (340b) has a pair of laterally-opposed notches (343) facing generally upwardly from lower hub portion (342b), and upper body portion (340a) has a pair of laterally-opposed edges (345) facing generally downwardly from upper hub portion (342a) and configured to mate with notches (343) for achieving proper alignment of body portions (340a, 340b). In some versions, one of notches (343) or edges (345) may include alignment pins and the other of notches (343) or edges (345) may include corresponding alignment bores for receiving the alignment pins. As best shown in FIG. 12, lower body portion (340b) also has a pair of laterally-opposed coupling wings (348) extending proximally from lower hub portion (342b). Body portions (340a, 340b) may each be constructed of any suitable translucent or opaque material, such as a polymeric material (e.g., plastic) or a metallic material. In the example shown, lower hub portion (342b) and wings (348) are integrally formed together with each other as a unitary (e.g., monolithic) piece to define lower body portion (340b). In this regard, body portions (340a, 340b) may each be manufactured via 3D printing, injection molding, investment casting, machining, and/or any other suitable manufacturing techniques. It will be appreciated that body portions (340a, 340b) may be either removably or permanently attached to each other in any suitable manner, such as via adhesive, thermal bonding, welding, snap fit, or any other attachment techniques.

Distal endoscope cap (310) of the present example also includes a generally cylindrical bore (350) extending longitudinally through lower hub portion (342b) and configured to be axially aligned with the working lumen of shaft assembly (108) when distal endoscope cap (310) is attached to open distal end (120), such that working element (101) may pass through bore (350) as working element (101) is guided through distal open end (120). While not shown, distal endoscope cap (310) may further include a navigation sensor assembly similar to navigation sensor assembly (152), which may be received within bore (350) or otherwise coupled to either body portion (340a, 340b) while permitting space for a working channel (354) to extend along bore (350) in a manner similar to that described above.

Distal endoscope cap (310) of the present example also includes an arch-shaped array of upper and lower generally rectangular bore portions (e.g., recesses) (360a, 360b, 362a, 362b) extending longitudinally along upper and lower hub portions (342a, 342b), respectively, configured to collectively define an array of generally rectangular bores similar to bores (160, 162) when upper and lower body portions (340a, 340b) are coupled to each other. More particularly, distal endoscope cap (310) includes an inner pair of upper bore portions (360a) extending radially outwardly from one or more lower, radially inner surfaces of upper body portion (340a), and an inner pair of lower bore portions (360b) extending radially inwardly from one or more upper, radially outer surfaces of lower body portion (340b), which are configured to collectively define an inner pair of laterally-opposed bores similar to bores (160) when upper and lower body portions (340a, 340b) are coupled to each other. Similarly, distal endoscope cap (310) includes an outer pair of upper bore portions (362a) extending radially outwardly from one or more lower, radially inner surfaces of upper body portion (340a), and an outer pair of lower bore portions (362b) extending radially inwardly from one or more upper, radially outer surfaces of lower body portion (340b), which are configured to collectively define an outer pair of laterally-opposed bores similar to bores (162) when upper and lower body portions (340a, 340b) are coupled to each other. While not shown, distal endoscope cap (310) may further include a pair of cameras similar to cameras (164), which may each be received within a respective inner bore defined by upper and lower inner bore portions (360a, 360b) in a manner similar to that described above, and/or a pair of illuminating elements similar to illuminating elements (166), which may each be received within a respective outer bore defined by upper and lower outer bore portions (362a, 362b) in a manner similar to that described above.

As shown, distal endoscope cap (310) of the present example also includes upper and lower irrigation lumen portions (e.g., recesses) (370a, 370b) extending longitudinally along upper and lower hub portions (342a, 342b), respectively, configured to collectively define an irrigation lumen when upper and lower body portions (340a, 340b) are coupled to each other. More particularly, distal endoscope cap (310) includes an upper irrigation lumen portion (370a) extending radially outwardly from one or more lower, radially inner surfaces of upper body portion (340a), and a lower irrigation lumen portion (370b) extending radially inwardly from one or more upper, radially outer surfaces of lower body portion (340b), which are configured to collectively define an irrigation lumen when upper and lower body portions (340a, 340b) are coupled to each other. In the example shown, irrigation lumen portions (370a, 370b) are each substantially semi-cylindrical, such that the irrigation lumen defined collectively thereby may be substantially cylindrical and may lack discrete fluid diversion passageways for diverging liquid into multiple streams and directing such streams laterally outwardly. Instead, front plate portion (340c) may be configured to provide such functionalities.

In this regard, and as best shown in FIG. 14, front plate portion (340c) of the present example includes a generally planar proximal surface (380), a top edge (382), and a bottom edge (384). Proximal surface (380) of front plate portion (340c) is configured to be positioned in-line with the irrigation lumen and is sized and shaped to substantially cover a distal end of the irrigation lumen while being disposed at least slightly distally thereof, such that substantially all liquid traveling distally through the irrigation lumen may impact proximal surface (380) to be laterally deflected by proximal surface (380). In this regard, distal endoscope cap (310) of the present example includes upper and lower shoulders (387, 388) (FIGS. 12-13) extending proximally from distal surfaces of upper and lower hub portions (342a, 342b), respectively, and disposed at least slightly distally relative to the respective irrigation lumen portions (370a, 370b), such that shoulders (387, 388) may collectively retain front plate portion (340c) at a predetermined position that is at least slightly distal of the irrigation lumen. In this manner, a pair of laterally-opposed fluid outlet slots (not shown) may be defined along the laterally-inner surfaces of the inner bores near distal ends thereof, between proximal surface (380) of front plate portion (340c) and the distal surfaces of body portions (340a, 340b) confronted thereby. Such fluid outlet slots may each direct a respective stream of liquid from the irrigation lumen laterally outwardly and/or proximally toward a distal end of a respective camera housed within a corresponding inner bore in a manner similar to that described above. In some versions, shoulders (387, 388) may collectively retain front plate portion (340c) such that a distal surface (not shown) of front plate portion (340c) may be substantially flush with the distal surfaces of upper and lower hub portions (342a, 342b).

### D. Distal Endoscope Cap Having Body with Three-Piece Construction Including Plug Portion

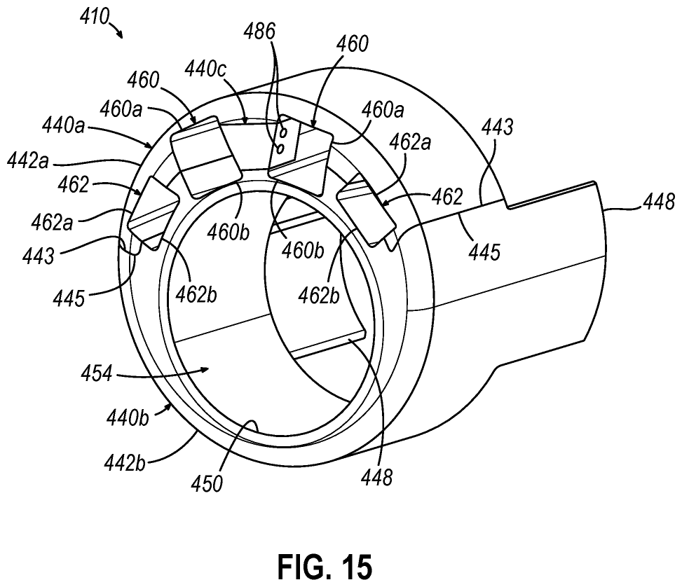

In some instances, it may be desirable to provide a distal endoscope cap having a body with a three-piece construction to facilitate or otherwise improve manufacturability of the distal endoscope cap, such as by enabling upper and lower portions of the irrigation lumen to be formed on upper and lower body portions, respectively, which may then be coupled to each other and to a fluid-diverting plug portion to define the body. In addition, or alternatively, such a three-piece construction of the body may enable upper and lower portions of the array of bores to be formed on the upper and lower body portions, respectively, so that the cameras and illuminating elements may be inserted into the lower portions of the respective bores, for example, and then covered by the upper body portion. FIGS. 15-19 show an example of a distal endoscope cap (410) having such functionalities, and which may be incorporated into instrument (100) in place of distal endoscope cap (110). Distal endoscope cap (410) may be similar to distal endoscope cap (110) described above except as otherwise described below. In this regard, distal endoscope cap (410) of the present example includes upper and lower body portions (440a, 440b) and a front plate portion (440c) configured to be coupled to each other to collectively define a body similar to body (140).

As shown, upper and lower body portions (440a, 440b) have upper and lower hub portions (442a, 442b), respectively, configured to collectively define a generally cylindrical hub (442) similar to hub (142) when upper and lower body portions (440a, 440b) are coupled to each other. To that end, lower body portion (440b) has a pair of laterally-opposed notches (443) facing generally upwardly from lower hub portion (442b), and upper body portion (440a) has a pair of laterally-opposed edges (445) facing generally downwardly from upper hub portion (442a) and configured to mate with notches (443) for achieving proper alignment of body portions (440a, 440b). In some versions, one of notches (443) or edges (445) may include alignment pins and the other of notches (443) or edges (445) may include corresponding alignment bores for receiving the alignment pins. In the example shown, lower body portion (440b) also has a pair of laterally-opposed coupling wings (448) extending proximally from lower hub portion (442b). Body portions (440a, 440b) may each be constructed of any suitable translucent or opaque material, such as a polymeric material (e.g., plastic) or a metallic material. In the example shown, lower hub portion (442b) and wings (448) are integrally formed together with each other as a unitary (e.g., monolithic) piece to define lower body portion (440b). In this regard, body portions (440a, 440b) may each be manufactured via 3D printing, injection molding, investment casting, machining, and/or any other suitable manufacturing techniques. It will be appreciated that body portions (440a, 440b) may be either removably or permanently attached to each other in any suitable manner, such as via adhesive, thermal bonding, welding, snap fit, or any other attachment techniques.

Distal endoscope cap (410) of the present example also includes a generally cylindrical bore (450) extending longitudinally through lower hub portion (442b) and configured to be axially aligned with the working lumen of shaft assembly (108) when distal endoscope cap (410) is attached to open distal end (120), such that working element (101) may pass through bore (450) as working element (101) is guided through distal open end (120). While not shown, distal endoscope cap (410) may further include a navigation sensor assembly similar to navigation sensor assembly (152), which may be received within bore (450) or otherwise coupled to either body portion (440a, 440b) while permitting space for a working channel (454) to extend along bore (450) in a manner similar to that described above.

Distal endoscope cap (410) of the present example also includes an arch-shaped array of upper and lower generally rectangular bore portions (e.g., recesses) (460a, 460b, 462a, 462b) extending longitudinally along upper and lower hub portions (442a, 442b), respectively, configured to collectively define an array of generally rectangular bores (460, 462) similar to bores (160, 162) when upper and lower body portions (440a, 440b) are coupled to each other. More particularly, distal endoscope cap (410) includes an inner pair of upper bore portions (460a) extending radially outwardly from one or more lower, radially inner surfaces of upper body portion (440a), and an inner pair of lower bore portions (460b) extending radially inwardly from one or more upper, radially outer surfaces of lower body portion (440b), which are configured to collectively define an inner pair of laterally-opposed bores (460) similar to bores (160) when upper and lower body portions (440a, 440b) are coupled to each other. Similarly, distal endoscope cap (410) includes an outer pair of upper bore portions (462a) extending radially outwardly from one or more lower, radially inner surfaces of upper body portion (440a), and an outer pair of lower bore portions (462b) extending radially inwardly from one or more upper, radially outer surfaces of lower body portion (440b), which are configured to collectively define an outer pair of laterally-opposed bores (462) similar to bores (162) when upper and lower body portions (440a, 440b) are coupled to each other. While not shown, distal endoscope cap (410) may further include a pair of cameras similar to cameras (164), which may each be received within a respective inner bore defined by upper and lower inner bore portions (460a, 460b) in a manner similar to that described above, and/or a pair of illuminating elements similar to illuminating elements (166), which may each be received within a respective outer bore defined by upper and lower outer bore portions (462a, 462b) in a manner similar to that described above.

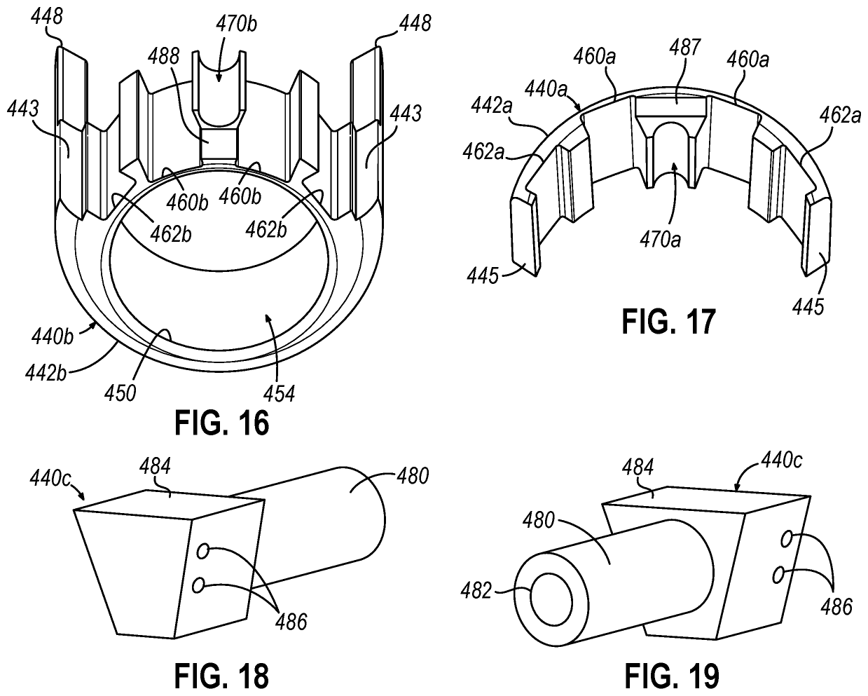

As best shown in FIGS. 16-17, distal endoscope cap (410) of the present example also includes upper and lower irrigation lumen portions (e.g., recesses) (470a, 470b) extending longitudinally along upper and lower hub portions (442a, 442b), respectively, configured to collectively define an irrigation lumen when upper and lower body portions (440a, 440b) are coupled to each other. More particularly, distal endoscope cap (410) includes an upper irrigation lumen portion (470a) extending radially outwardly from one or more lower, radially inner surfaces of upper body portion (440a), and a lower irrigation lumen portion (470b) extending radially inwardly from one or more upper, radially outer surfaces of lower body portion (440b), which are configured to collectively define an irrigation lumen when upper and lower body portions (440a, 440b) are coupled to each other. In the example shown, irrigation lumen portions (470a, 470b) are each substantially semi-cylindrical, such that the irrigation lumen defined collectively thereby may be substantially cylindrical and may lack discrete fluid diversion passageways for diverging liquid into multiple streams and directing such streams laterally outwardly. Instead, front plug portion (440c) may be configured to provide such functionalities.

In this regard, and as best shown in FIGS. 18-19, front plug portion (440c) of the present example includes a generally cylindrical proximal conduit (480) having a fluid inlet bore (482), and a distal manifold (484) having a plurality of fluid outlet bores (486). Proximal conduit (480) of front plug portion (440c) is configured to be coaxial with and at least partially received within the irrigation lumen, such that substantially all liquid traveling distally through the irrigation lumen may be diverted into multiple (e.g., four) divergent streams within distal manifold (484), which may then be directed laterally outwardly and/or proximally by respective fluid outlet bores (486) toward a distal end of a respective camera housed within a corresponding inner bore (460) in a manner similar to that described above. Distal endoscope cap (410) of the present example includes upper and lower ledges (487, 488) (FIGS. 16-17) extending proximally from distal surfaces of upper and lower hub portions (442a, 442b), respectively, such that ledges (487, 488) may collectively retain distal manifold (484) of front plug portion (440c) and thereby allow a distal surface of distal manifold (484) to be substantially flush with the distal surfaces of upper and lower hub portions (442a, 442b).

### E. Example of a Distal Endoscope Cap with Proximal Coupling Collar

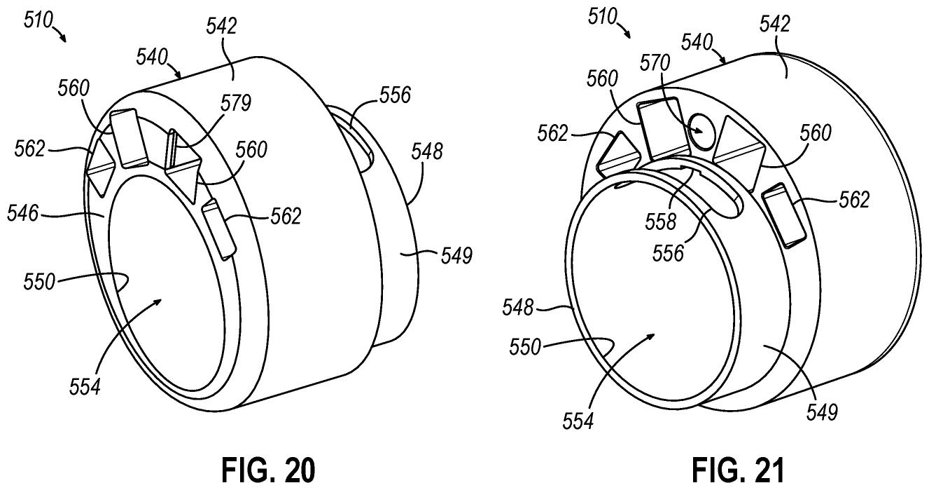

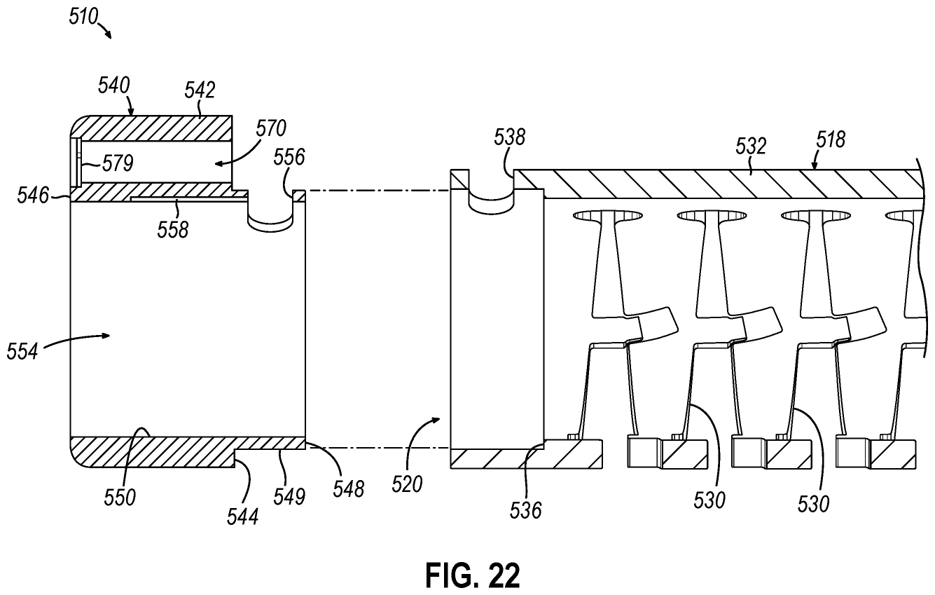

In some instances, it may be desirable to provide a distal endoscope cap having a collar for engaging an inner surface of flexible portion (118) for facilitating attachment of the distal endoscope cap thereto. FIGS. 20-22 show an example of a distal endoscope cap (510) and a flexible portion (518) (FIG. 22) having such functionalities, and which may be incorporated into instrument (100) in place of distal endoscope cap (110) and flexible portion (118), respectively. Distal endoscope cap (510) and flexible portion (518) may be similar to distal endoscope cap (110) and flexible portion (118) described above, respectively, except as otherwise described below. In this regard, flexible portion (518) of the present example extends distally to an open distal end (520) and includes a linear array of articulating ribs (530) connected to each other by a resilient spine (532). As shown, flexible portion (518) includes a distal counterbore (536) extending proximally from open distal end (520) for facilitating coupling of distal endoscope cap (510) to open distal end (520). In some versions, distal counterbore (536) may have a diameter of about 3.1 mm. Alternatively, distal counterbore (536) may have any other suitable diameter. Flexible portion (518) also includes a slot (538) extending radially between distal counterbore (536) and a generally cylindrical outer surface of flexible portion (518) and generally angularly aligned with resilient spine (532) about a longitudinal axis of flexible portion (518), the purpose of which is described below.

Distal endoscope cap (510) of the present example includes a body (540) having a generally cylindrical hub (542) extending between proximal and distal surfaces (544, 546). As shown, body (540) also has a generally cylindrical coupling collar (548) extending proximally from proximal surface (544) of hub (542). Body (540) may be constructed of any suitable translucent or opaque material, such as a polymeric material (e.g., plastic) or a metallic material. In the example shown, hub (542) and collar (548) are integrally formed together with each other as a unitary (e.g., monolithic) piece to define body (540). In this regard, body (540) may be manufactured via 3D printing, injection molding, investment casting, machining, and/or any other suitable manufacturing techniques.

As shown, collar (548) of distal endoscope cap (510) includes a generally cylindrical outer gripping surface (549) configured to frictionally engage a generally cylindrical inner surface of flexible portion (518) near open distal end (520), such as distal counterbore (536), for removably attaching distal endoscope cap (510) to open distal end (520). In the example shown, gripping surface (549) has a diameter substantially equal to or slightly greater than the diameter of distal counterbore (536) for enhancing the frictional engagement between gripping surface (549) and distal counterbore (536). In some versions, collar (548) may be configured to flex slightly radially inwardly during insertion of collar (548) into distal counterbore (536). In addition, or alternatively, proximal surface (544) of hub (542) may be configured to abut a distal end of flexible portion (518) during insertion of collar (548) into distal counterbore (536) to provide a positive stop therebetween. While gripping surface (549) of collar (548) is shown frictionally engaging distal counterbore (536) of flexible portion (518), it will be appreciated that distal endoscope cap (510) may be either removably or permanently attached to open distal end (520) in any suitable manner, such as via adhesive, thermal bonding, welding, snap fit, or any other attachment techniques.

Distal endoscope cap (510) of the present example also includes a generally cylindrical bore (550) extending longitudinally between a proximal surface of collar (548) and distal surface (546) of hub (542) and configured to be axially aligned with the working lumen of shaft assembly (108) when distal endoscope cap (510) is attached to open distal end (120), such that working element (101) may pass through bore (550) as working element (101) is guided through distal open end (120). While not shown, distal endoscope cap (510) may further include a navigation sensor assembly similar to navigation sensor assembly (152), which may be received within bore (550) or otherwise coupled to body (540) while permitting space for a working channel (554) to extend along bore (550) in a manner similar to that described above. In this regard, distal endoscope cap (510) includes a slot (556) extending radially between bore (550) and outer gripping surface (549) for facilitating routing of such wires to the navigation sensor assembly laterally through collar (548) into bore (550) when the navigation sensor assembly is received within bore (550). As shown in FIG. 22, slot (556) of distal endoscope cap (510) may be generally angularly aligned with resilient slot (538) of flexible portion (518) about the longitudinal axis of flexible portion (518) for facilitating routing of such wires along resilient spine (132) and laterally through flexible portion (518) into slot (556). In the example shown, distal endoscope cap (510) further includes an elongate recess (558) extending distally from slot (556) along bore (550) for facilitating routing of such wires longitudinally therealong to the navigation sensor assembly. In addition, or alternatively, recess (558) may receive one or more solder pads and/or wire connections for coupling such wires to the navigation sensor assembly.

Distal endoscope cap (510) of the present example also includes an arch-shaped array of generally rectangular bores (560, 562) each extending longitudinally between proximal and distal surfaces (544, 546) of hub (542) and disposed about (e.g., above) bore (550). More particularly, distal endoscope cap (510) includes an inner pair of laterally-opposed bores (560) and an outer pair of laterally-opposed bores (562). While not shown, distal endoscope cap (510) may further include a pair of cameras similar to cameras (164), which may each be received within a respective inner bore (560) in a manner similar to that described above, and/or a pair of illuminating elements similar to illuminating elements (166), which may each be received within a respective outer bore (562) in a manner similar to that described above.

As shown, distal endoscope cap (510) of the present example also includes an irrigation lumen (570) extending distally from proximal surface (544) toward distal surface (546) of hub (542) and disposed above bore (550) between inner bores (560) for directing streams of liquid generally laterally outwardly into inner bores (560) through fluid outlet slots (579) in a manner similar to that described above.

### F. Example of a Distal Endoscope Cap with Single Camera, Single Illuminating Element, and Two Navigation Sensors

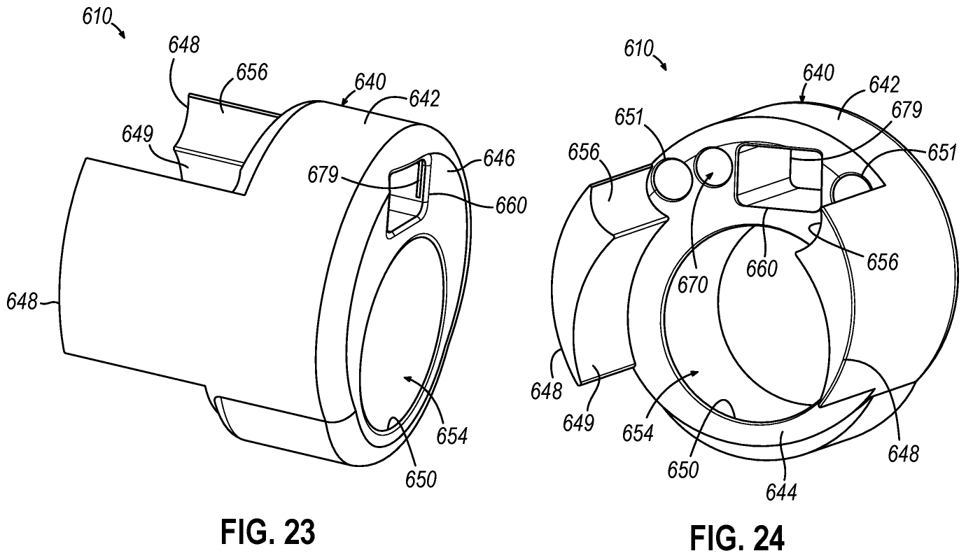

In some instances, it may be desirable to provide a distal endoscope cap with a single camera and/or single illuminating element, such as for reducing the size of the distal endoscope cap. In addition, or alternatively, it may be desirable to provide a distal endoscope cap with two single-axis sensors, such as for positioning the single-axis sensors outside of the working channel and thereby increasing the size of the working channel. FIGS. 23-24 show an example of a distal endoscope cap (610) having such functionalities, and which may be incorporated into instrument (100) in place of distal endoscope cap (110). Distal endoscope cap (610) may be similar to distal endoscope cap (110) described above except as otherwise described below. In this regard, distal endoscope cap (610) of the present example includes a body (640) having a generally cylindrical hub (642) extending between proximal and distal surfaces (644, 646).

As shown, body (640) has a pair of laterally-opposed coupling wings (648) extending proximally from proximal surface (644) of hub (642). Body (640) may be constructed of any suitable translucent or opaque material, such as a polymeric material (e.g., plastic) or a metallic material. In the example shown, hub (642) and wings (648) are integrally formed together with each other as a unitary (e.g., monolithic) piece to define body (640). In this regard, body (640) may be manufactured via 3D printing, injection molding, investment casting, machining, and/or any other suitable manufacturing techniques.

In the example shown, each wing (648) of distal endoscope cap (610) includes a laterally inwardly-facing gripping surface (649) configured to frictionally engage a generally cylindrical outer surface of flexible portion (118) of shaft assembly (108) near open distal end (120) for removably attaching distal endoscope cap (610) to open distal end (120) in a manner similar to that described above.

Distal endoscope cap (610) of the present example also includes a generally cylindrical bore (650) extending longitudinally between proximal and distal surfaces (644, 646) of hub (642) and configured to be axially aligned with the working lumen of shaft assembly (108) when distal endoscope cap (610) is attached to open distal end (120), such that working element (101) may pass through bore (650) as working element (101) is guided through distal open end (120). Thus, a working channel (654) may extend along bore (650).

As shown in FIG. 24, distal endoscope cap (610) of the present example also includes an outer pair of laterally-opposed, generally cylindrical bores (651) each extending distally from proximal surface (644) toward distal surface (646) of hub (642) and disposed above bore (650). While not shown, distal endoscope cap (610) may further include a pair of navigation sensors, which may be received within respective bore (651). In some versions, each such navigation sensor may be configured as a single-axis sensor. In this regard, distal endoscope cap (610) includes a pair of laterally-opposed grooves (656) extending along respective wings (648) above the corresponding gripping surfaces (649) for facilitating routing of wires to the respective navigation sensors. In addition, or alternatively, each of the navigation sensors may be similar to navigation sensor assembly (152).

Distal endoscope cap (610) of the present example also includes an inner generally rectangular bore (660) extending longitudinally between proximal and distal surfaces (644, 646) of hub (642) and disposed above bore (650) and between inner bores (651). While not shown, distal endoscope cap (610) may further include a single camera similar to cameras (164), which may be received within a first region of bore (660) in a manner similar to that described above, and a single illuminating element similar to illuminating elements (166), which may be received within a second region of bore (660) (e.g., above, below, or adjacent to the first region) in a manner similar to that described above.

As shown, distal endoscope cap (610) of the present example also includes an irrigation lumen (670) extending distally from proximal surface (644) toward distal surface (646) of hub (642) and disposed above bore (650) along one lateral side of inner bore (660) for directing a stream of liquid generally laterally inwardly into inner bore (660) through a fluid outlet slot (679) in a manner similar to that described above.

### G. Example of a Distal Endoscope Cap with Two Cameras, Single Illuminating Element, and Two Irrigation Fluid Conduit Grooves

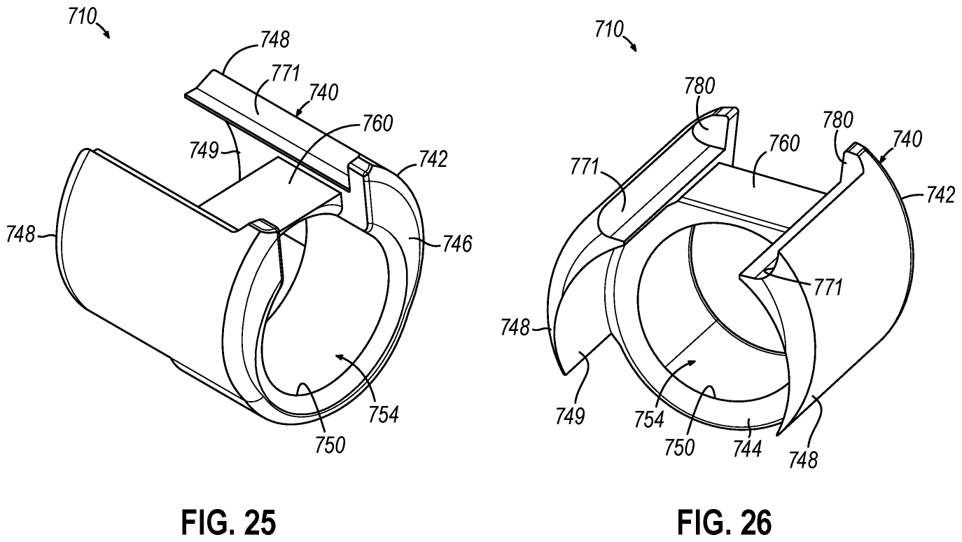

In some instances, it may be desirable to provide a distal endoscope cap with two cameras and two corresponding irrigation fluid conduits, such as for independently cleaning the distal ends of the cameras. FIGS. 25-26 show an example of a distal endoscope cap (710) having such functionalities, and which may be incorporated into instrument (100) in place of distal endoscope cap (110). Distal endoscope cap (710) may be similar to distal endoscope cap (110) described above except as otherwise described below. In this regard, distal endoscope cap (710) of the present example includes a body (740) having a generally cylindrical hub (742) extending between proximal and distal surfaces (744, 746).

As shown, body (740) has a pair of laterally-opposed coupling wings (748) extending proximally from proximal surface (744) of hub (742). Body (740) may be constructed of any suitable translucent or opaque material, such as a polymeric material (e.g., plastic) or a metallic material. In the example shown, hub (742) and wings (748) are integrally formed together with each other as a unitary (e.g., monolithic) piece to define body (740). In this regard, body (740) may be manufactured via 3D printing, injection molding, investment casting, machining, and/or any other suitable manufacturing techniques.

In the example shown, each wing (748) of distal endoscope cap (710) includes a laterally inwardly-facing gripping surface (749) configured to frictionally engage a generally cylindrical outer surface of flexible portion (118) of shaft assembly (108) near open distal end (120) for removably attaching distal endoscope cap (710) to open distal end (120) in a manner similar to that described above.

Distal endoscope cap (710) of the present example also includes a generally cylindrical bore (750) extending longitudinally between proximal and distal surfaces (744, 746) of hub (742) and configured to be axially aligned with the working lumen of shaft assembly (108) when distal endoscope cap (710) is attached to open distal end (120), such that working element (101) may pass through bore (750) as working element (101) is guided through distal open end (120). While not shown, distal endoscope cap (710) may further include a navigation sensor assembly similar to navigation sensor assembly (152), which may be received within bore (750) or otherwise coupled to body (740) while permitting space for a working channel (754) to extend along bore (750) in a manner similar to that described above.

Distal endoscope cap (710) of the present example also includes a platform (760) extending distally from proximal surface (744) toward distal surface (746) of hub (742) and disposed above bore (750). While not shown, distal endoscope cap (710) may further include a pair of cameras similar to cameras (164), which may be positioned atop laterally outer regions of platform (760), and a single illuminating element similar to illuminating elements (166), which may be positioned atop a laterally inner region of platform (760) between the pair of cameras.

As shown, distal endoscope cap (710) of the present example also includes a pair of laterally-opposed grooves (771) extending along respective wings (748) above the corresponding gripping surfaces (749) for receiving corresponding irrigation fluid conduits (not shown). In this regard, distal endoscope cap (710) includes a pair of laterally-opposed, proximally-facing fluid diversion surfaces (780) each configured to be positioned in-line with the corresponding irrigation fluid conduit and sized and shaped to substantially cover a distal end of the corresponding irrigation fluid conduit while being disposed at least slightly distally thereof, such that substantially all liquid traveling distally through each irrigation fluid conduit may impact the corresponding fluid diversion surface (780) to be deflected laterally inwardly and/or proximally by the corresponding fluid diversion surface (780) toward a distal end of a respective camera positioned atop platform (760).

### H. Example of a Dual-Lumen Routing Device

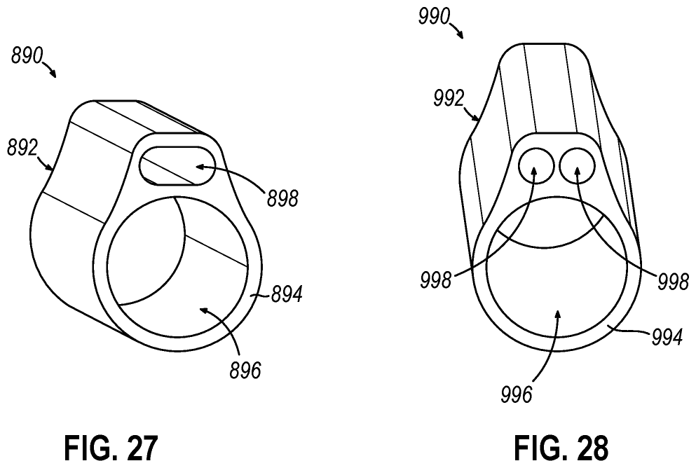

In some instances, it may be desirable to provide a routing device for directing an irrigation fluid conduit together with wires along a flexible guide shaft, such as any of flexible portions (118, 518) of shaft assembly (108) of instrument (100), to a distal endoscope cap, such as any of distal endoscope caps (110, 210, 310, 410, 510, 610, 710, 1410, 1510). FIG. 27 shows an example of a routing device (890) having such functionalities.

Routing device (890) of the present example includes a generally pear-shaped body (892) extending between a proximal surface (894) and a distal surface (not shown). Body (892) may be constructed of any suitable material, such as a polymeric material (e.g., plastic) or a metallic material, and may be manufactured via 3D printing, injection molding, investment casting, machining, extruding and/or any other suitable manufacturing techniques. In some versions, body (892) may be constructed of a flexible material to accommodate bending of flexible portion (118, 518), as described in greater detail below.

Routing device (890) of the present example also includes a generally cylindrical lumen (896) extending longitudinally between proximal surface (894) and the distal surface of body (892) and configured to frictionally engage a generally cylindrical outer surface of flexible portion (118, 518) of shaft assembly (108) for securing routing device (890) thereto. For example, lumen (896) may have a diameter substantially equal to or slightly less than a diameter of the cylindrical outer surface of flexible portion (118, 518) for enhancing the frictional engagement between lumen (896) and the cylindrical outer surface of flexible portion (118, 518).

Routing device (890) of the present example also includes a generally obround lumen (898) extending longitudinally between proximal surface (894) and the distal surface of body (892) above lumen (896). Lumen (898) may be configured to direct an irrigation fluid conduit together with wires (not shown) along flexible portion (118, 518) to distal endoscope cap (110, 210, 310, 410, 510, 610, 710, 1410, 1510). In cases where the illuminating elements of distal endoscope cap (110, 210, 310, 410, 510, 610, 710, 1410, 1510) include fiber optic components, one or more respective optical fibers may also be directed through lumen (898) for routing along flexible portion (118, 518). In some versions, lumen (898) may be generally angularly aligned with resilient spine (132, 532) of flexible portion (118, 518) of shaft assembly (108) about the longitudinal axis of flexible portion (118, 518) for facilitating routing of the irrigation fluid conduit, wires, and/or optical fibers along resilient spine (132, 532).

In some versions, routing device (890) may have a length substantially equal to or greater that of flexible portion (118, 518) such that routing device (890) may extend along substantially the entire length of flexible portion (118, 518). In such cases, body (892) of routing device (890) may be constructed of a material having sufficient flexibility to avoid interfering with the ability of flexible portion (118, 518) to bend. Such versions may enable lumen (898) to serve as the irrigation fluid conduit by enabling liquid to flow directly through lumen (898) (e.g., around the wires and/or optical fibers). In other versions, routing device (890) may have a length substantially equal to or less than that of each articulating rib (130, 530) such that a plurality of routing devices (890) may be secured to corresponding articulating ribs (130, 530) along the length of flexible portion (118, 518), and may thereby avoid interfering with the ability of flexible portion (118, 518) to bend.

### I. EXAMPLE OF A TRIPLE-LUMEN ROUTING DEVICE

In some instances, it may be desirable to provide a routing device for directing an irrigation fluid conduit and wires separately from each other along a flexible guide shaft, such as any of flexible portions (118, 518) of shaft assembly (108) of instrument (100), to a distal endoscope cap, such as any of distal endoscope caps (110, 210, 310, 410, 510, 610, 710, 1410, 1510). FIG. 28 shows an example of a routing device (990) having such functionalities, and which may be incorporated into instrument (100) in place of routing device (890). Routing device (990) may be similar to routing device (890) described above except as otherwise described below. In this regard, routing device (990) of the present example includes a generally pear-shaped body (992) extending between a proximal surface (994) and a distal surface (not shown), and further includes a generally cylindrical lumen (996) extending longitudinally between proximal surface (994) and the distal surface of body (992) and configured to frictionally engage a generally cylindrical outer surface of flexible portion (118, 518) of shaft assembly (108) for securing routing device (990) thereto.

As shown, routing device (990) of the present example also includes a pair of generally cylindrical lumens (998) extending longitudinally between proximal surface (994) and the distal surface of body (992) above lumen (996). One lumen (998) may be configured to direct an irrigation fluid conduit (not shown) along flexible portion (118, 518) to distal endoscope cap (110, 210, 310, 410, 510, 610, 710, 1410, 1510), while the other lumen (998) may be configured to direct wires (not shown) along flexible portion (118, 518) to distal endoscope cap (110, 210, 310, 410, 510, 610, 710, 1410, 1510). In cases where the illuminating elements of distal endoscope cap (110, 210, 310, 410, 510, 610, 710, 1410, 1510) include fiber optic components, one or more respective optical fibers may also be directed through the other lumen (898) (e.g., together with the wires) for routing along flexible portion (118, 518). In some versions, lumens (998) may each be generally angularly aligned with resilient spine (132, 532) of flexible portion (118, 518) of shaft assembly (108) about the longitudinal axis of flexible portion (118, 518) for facilitating routing of the irrigation fluid conduit, wires, and/or optical fibers along resilient spine (132, 532).

In some versions, routing device (990) may have a length substantially equal to or greater that of flexible portion (118, 518) such that routing device (990) may extend along substantially the entire length of flexible portion (118, 518). In such cases, body (992) of routing device (990) may be constructed of a material having sufficient flexibility to avoid interfering with the ability of flexible portion (118, 518) to bend. Such versions may enable one lumen (998) to serve as the irrigation fluid conduit by enabling liquid to flow directly therethrough, while the wires and/or optical fibers are separately housed the other lumen (998). In other versions, routing device (990) may have a length substantially equal to or less than that of each articulating rib (130, 530) such that a plurality of routing devices (990) may be secured to corresponding articulating ribs (130, 530) along the length of flexible portion (118, 518), and may thereby avoid interfering with the ability of flexible portion (118, 518) to bend.

### III. EXAMPLES OF AN ENT INSTRUMENT WITH ERGONOMIC HANDLE AND CABLE MANAGEMENT FEATURES

In some instances, it may be desirable to provide an ENT instrument having a handle assembly with a body that rotates together with the shaft assembly such that the operator may grasp and rotate the body with a single hand to rotate the shaft assembly about its longitudinal axis, thereby allowing rotation control knob (124) to be omitted. In addition, or alternatively, it may be desirable to equip an ENT instrument with a strain relief for protecting the various cables or conduits (e.g., irrigation fluid tube(s), suction tube(s), electrical wire(s), and/or optical fiber(s)) proximally exiting the handle assembly from mechanical stress during rotation of the body of the handle assembly about the longitudinal axis of the shaft assembly and/or for allowing such rotation of the body of the handle assembly to be substantially unencumbered by such cables or conduits, at least within a predetermined range of motion. Each of the examples of ENT instrument (1100) and handle assemblies (1106, 1206) described below may function in such a manner.

### A. Example of an Instrument with Handle Assembly Having Deflection Control Knob Coupled to Leadscrew for Actuating Deflection of Distal Shaft End

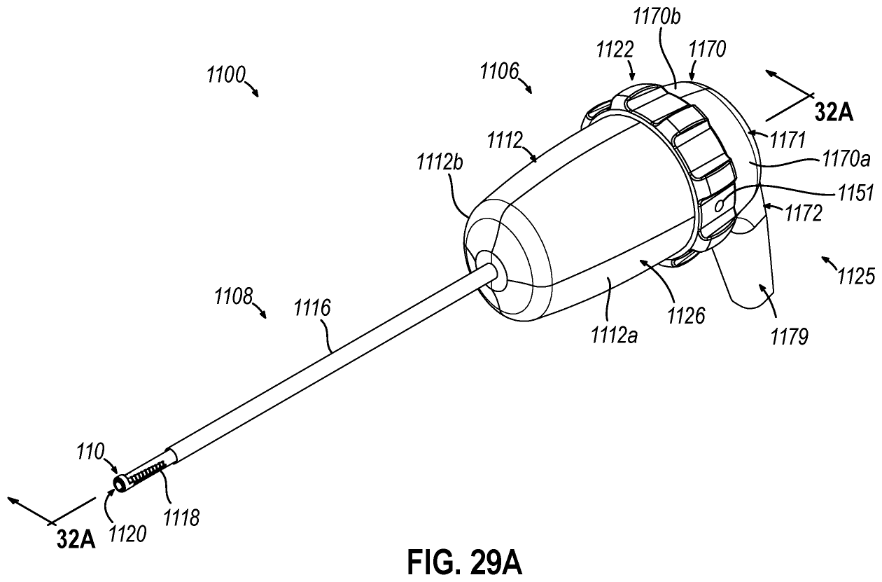

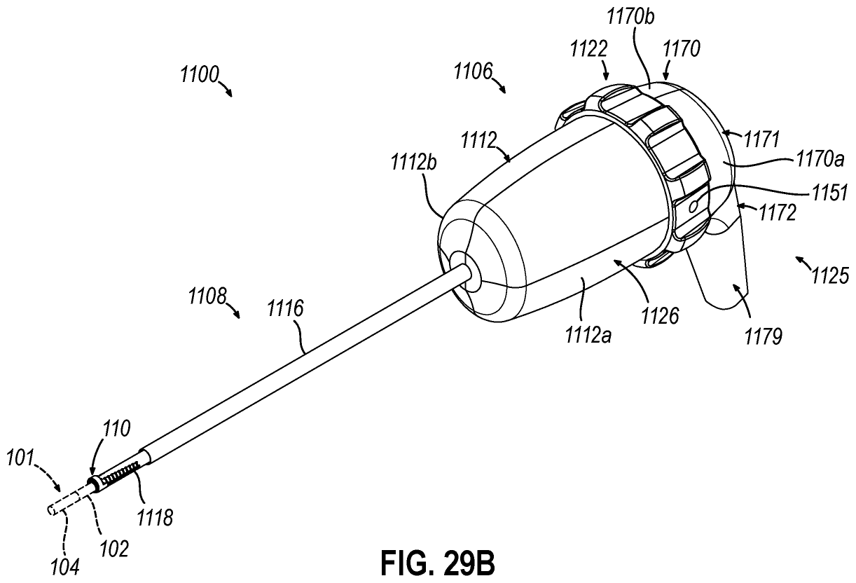

FIGS. 29A-32B show another example of an instrument (1100) to which distal endoscope cap (110) may be either removably or permanently attached for providing instrument (1100) with visualization and/or navigation capabilities. While distal endoscope cap (110) is shown, it will be appreciated that any other distal endoscope cap, such as any of distal endoscope caps (210, 310, 410, 510, 610, 710, 1410, 1510), may be used in place of distal endoscope cap (110).

Instrument (1100) is similar to instrument (100) described above, except as otherwise described below. In this regard, instrument (1100) may be used to guide working element (101) (FIG. 29B) into an anatomical passageway, and includes a handle assembly (1106) and a shaft assembly (1108). Instrument (1100) may be coupled with an inflation fluid source (not shown), which may be operable to selectively supply an inflation fluid to a balloon (not shown) of end effector (104), for inflating the balloon to thereby dilate the anatomical passageway. In addition, or alternatively, instrument (1100) may be coupled with an ablation energy generator (not shown), which may be operable to generate ablation energy for delivery to tissue via electrodes (not shown) of end effector (104)) to thereby ablate, electroporate, or apply resistive heating to the tissue. Energy produced by the ablation energy generator may include, but is not limited to, radiofrequency (RF) energy or pulsed-field ablation (PFA) energy, including monopolar or bipolar high-voltage DC pulses as may be used to effect irreversible electroporation (IRE), or combinations thereof.

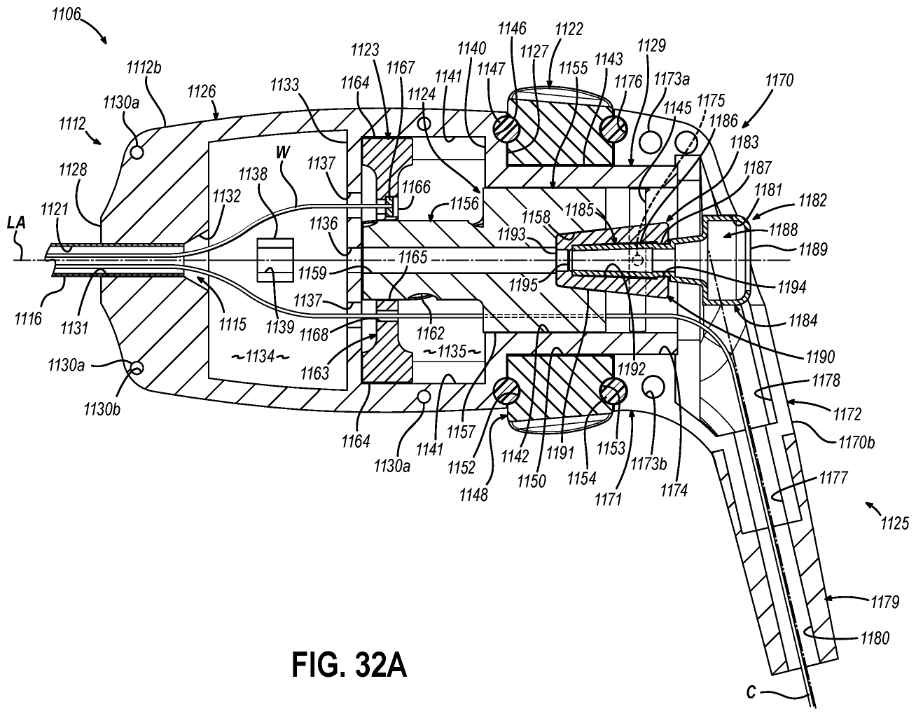

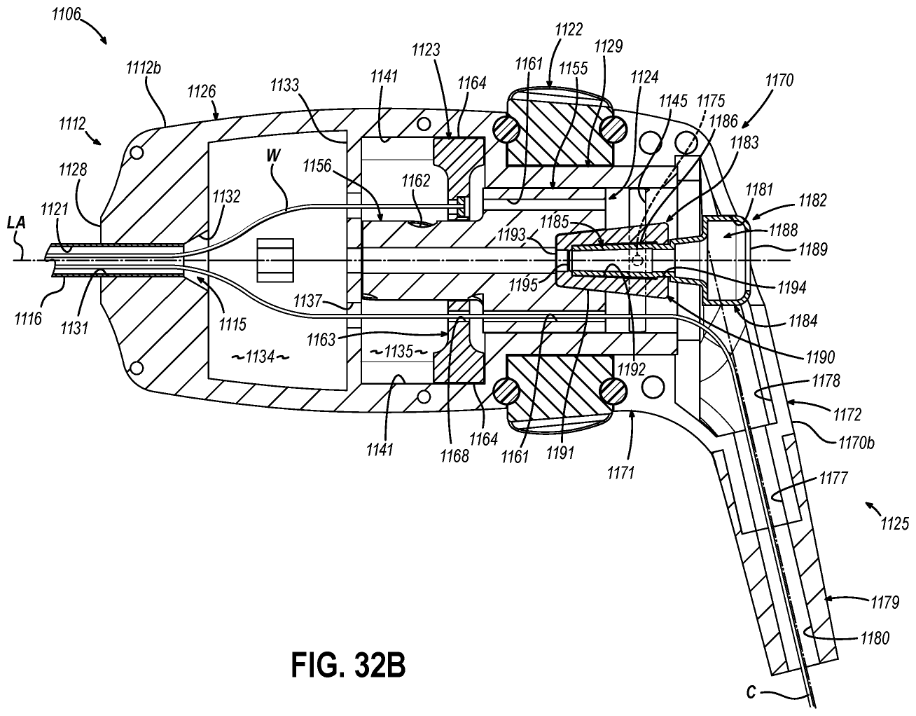

Handle assembly (1106) of this example includes a rigid handle body (1112) that is sized and configured to be grasped and operated by a single hand of an operator (e.g., either a left hand or a right hand), such as via a power grip, a pencil grip, or any other suitable kind of grip. In some versions, handle assembly (1106) may have a length of between about 9 cm and about 11 cm. Shaft assembly (1108) of the present example includes an open proximal end (1115) (FIGS. 32A-32B), a rigid portion (1116), a flexible portion (1118) distal to rigid portion (1116), and an open distal end (1120). Shaft assembly (1108) of the present example is non-rotatable (e.g., fixed against rotation) relative to handle body (1112), such that shaft assembly (1108) and handle body (1112) may be collectively rotatable about the longitudinal axis (LA) of rigid portion (1116), as described in greater detail below. Flexible portion (also referred to as a flexible guide shaft or a deflectable guide shaft) (1118) of shaft assembly (1108) is similar to flexible portion (118) of shaft assembly (108) described above. A working lumen (1121) (FIGS. 32A-32B) extends longitudinally from open proximal end (1115) of shaft assembly (1108) all the way to open distal end (1120) and is configured to slidably receive working element (101), such that shaft assembly (1108) may receive working element (101) at open proximal end (1115), and such that shaft assembly (1108) may guide working element (101) out through open distal end (1120). In some versions, working lumen (1121) may have a diameter of about 2.9 mm.

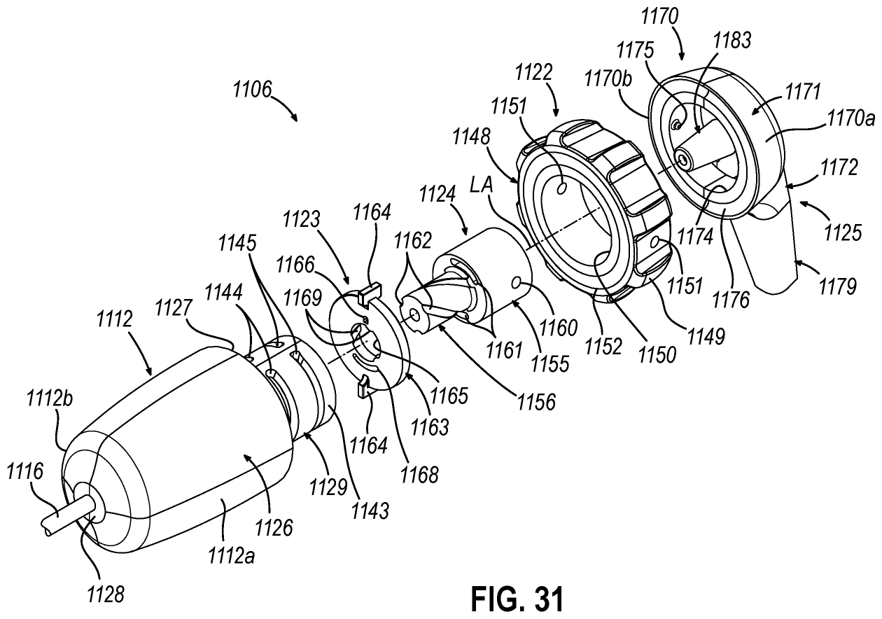

A distal end of a pull-wire (W) (FIGS. 32A-32B) is coupled with flexible portion (1118) in a manner similar to that described above, and a proximal end of pull-wire (W) is operatively coupled with a deflection control knob (1122) of handle assembly (1106). Deflection control knob (1122) is rotatable relative to handle body (1112), about the longitudinal axis (LA) of shaft assembly (1108), to selectively retract pull-wire (W) proximally. As pull-wire (W) is retracted proximally, flexible portion (1118) bends and thereby deflects distal end (1120) laterally away from the longitudinal axis (LA) of rigid portion (1116). In this regard, the proximal end of pull-wire (W) is fixedly secured to a linear output member of a linear actuator in the form of a lead nut (1123) (FIG. 31) that is slidably housed within handle body (1112) and configured to translate along the longitudinal axis (LA) between a distal position (FIG. 32A) and a proximal position (FIG. 32B) in response to rotation of deflection control knob (1122) about the longitudinal axis (LA), via a rotary input member of the linear actuator in the form of a leadscrew (1124) (FIG. 31). Deflection control knob (1122), lead nut (1123), leadscrew (1124), pull-wire (W), and flexible portion (1118) thus cooperate to impart steerability to shaft assembly (1108), as described in greater detail below.

Handle assembly (1106) of the present example further includes a strain relief (also referred to as a cable guide) (1125) rotatably coupled to handle body (1112) proximal of deflection control knob (1122). Strain relief (1125) is rotatable relative to handle body (1112), independently of deflection control knob (1122), about the longitudinal axis (LA) of shaft assembly (1108) to reorient the proximal portion of at least one conduit (C) (FIGS. 32A-32B) proximally exiting handle assembly (1106) relative to the longitudinal axis (LA). Conduit (C) may include any one or more irrigation fluid tube(s), suction tube(s), electrical wire(s), and/or optical fiber(s) that may extend distally through shaft assembly (1108) (e.g., parallel to the longitudinal axis (LA)). For example, conduit (C) may include an irrigation fluid tube extending distally to inlet port (172) of distal endoscope cap (110) for placing irritation lumen (170) of distal endoscope cap (110) in fluid communication with a source of irrigation fluid (e.g., saline or any other suitable liquid). In addition, or alternatively, conduit (C) may include one or more wire(s) extending distally to camera leads (167) and/or illuminating element leads (169) of distal endoscope cap (110) for operatively coupling camera leads (167) and/or illuminating element leads (169) to the coupling unit. As another example, conduit (C) may include one or more optical fiber(s) extending distally to one or more lenses of illuminating elements (166) of distal endoscope cap (110) for optically coupling the one or more lenses with a proximal source of light.

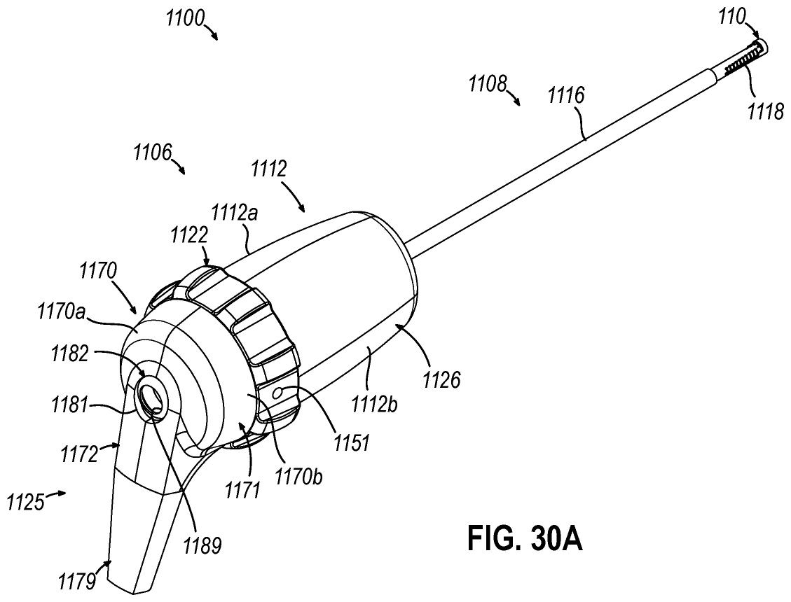

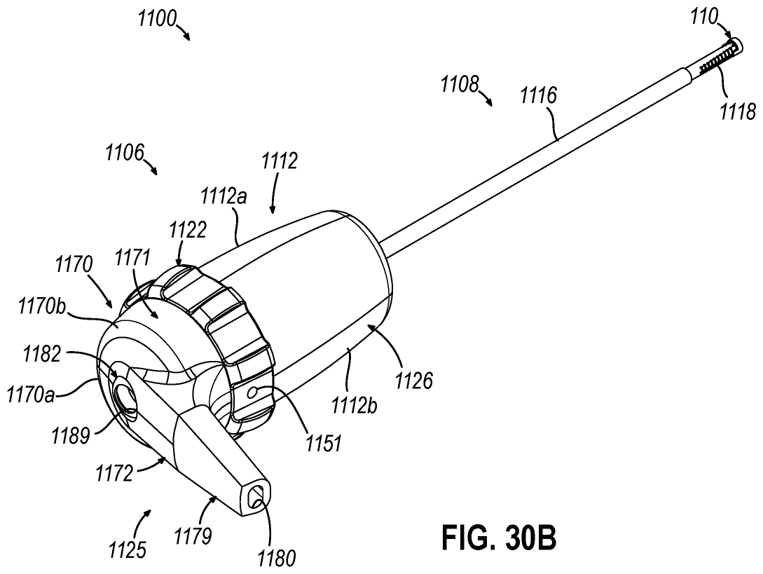

As shown in FIGS. 30A-30B, strain relief (1125) is rotatable between a first orientation about the longitudinal axis (LA) of shaft assembly (1108) in which strain relief (1125) is configured to direct the proximal portion of conduit (C) away from the longitudinal axis (LA) in a first direction (FIG. 30A), and a second orientation about the longitudinal axis (LA) in which strain relief (1125) is configured to direct the proximal portion of conduit (C) away from the longitudinal axis (LA) in a second direction (FIG. 30B). In the example shown, strain relief (1125) is rotatable within a range of motion of at least 90° such that the first direction is generally transversely downward relative to the longitudinal axis (LA) and the second direction is generally laterally rightward relative to the longitudinal axis (LA) in the frame of reference of FIGS. 30A-30B. In some versions, strain relief (1125) is rotatable within a range of motion of at least about 180°. For example, strain relief (1125) may be rotatable from the first orientation beyond the second orientation to a third orientation in which strain relief (1125) is configured to direct the proximal portion of conduit (C) away from the longitudinal axis (LA) in a third direction that is generally transversely upward relative to the longitudinal axis (LA) in the frame of reference of FIGS. 30A-30B. In addition, or alternatively, strain relief (1125) may be rotatable from the second orientation beyond the first orientation to a fourth orientation in which strain relief (1125) is configured to direct the proximal portion of conduit (C) away from the longitudinal axis (LA) in a fourth direction that is generally laterally leftward relative to the longitudinal axis (LA) in the frame of reference of FIGS. 30A-30B.

Referring now primarily to FIGS. 32A-32B, and with continuing reference to FIG. 31, handle body (1112) of the present example includes a generally cylindrical hub (1126) extending between proximal and distal surfaces (1127, 1128), and a generally cylindrical collar (1129) extending proximally from proximal surface (1127) of hub (1126). In the example shown, handle body (1112) is formed from first and second shells (e.g., halves) (1112a, 1112b) fixedly coupled to each other along a longitudinal centerline of handle body (1112) via corresponding pairs of engagement features in the form of detents (1130a) and indents (1130b), such that shells (1112a, 1112b) collectively define handle body (1112). Shells (1112a, 1112b) may each be manufactured via 3D printing, injection molding, investment casting, machining, and/or any other suitable manufacturing techniques, and subsequently assembled to each other to form handle body (1112). It will be appreciated that handle body (1112) may be formed in any other suitable manner. For example, shells (1112a, 1112b) may be integrally formed together with each other as a unitary (e.g., monolithic) piece to define handle body (1112), such that detents (1130a) and indents (1130b) may be omitted.

Handle body (1112) of the present example also includes a generally cylindrical distal bore (1131) extending proximally from distal surface (1128) of hub (1126) and configured to receive rigid portion (1116) of shaft assembly (1108) for fixedly securing rigid portion (1116) to handle body (1112), such that distal bore (1131) extends along the longitudinal axis (LA) of rigid portion (1116). For example, distal bore (1131) may be configured to frictionally engage a generally cylindrical outer surface of rigid portion (1116) of shaft assembly (1108) for fixedly securing rigid portion (1116) to handle body (1112). In the example shown, a generally frustoconical bore (1132) extends proximally and tapers radially outwardly from distal bore (1131) for directing pull-wire (W) and conduit (C) proximally out of rigid portion (1116) at or near open proximal end (1115) and away from the longitudinal axis (LA) and/or for funneling working element (101) into open proximal end (1115).

Hub (1126) of the present example also includes an internal partition (1133) extending laterally and/or transversely across a hollow interior of hub (1126) to divide the interior of hub (1126) into a distal interior chamber (1134) and a proximal interior chamber (1135). As shown, distal chamber (1134) extends longitudinally between frustoconical bore (1132) and partition (1133), and is sized and shaped to facilitate insertion of working element (101) and/or one or more suction components (not shown) therethrough, and to permit pull-wire (W) and conduit (C) to exit proximally out of rigid portion (1116) along respective paths away from the longitudinal axis (LA). In this regard, a generally cylindrical central bore (1136) extends through partition (1133) along the longitudinal axis (LA) for permitting passage of working element (101) therethrough into distal chamber (1134), and a pair of transversely-opposed, elongate slots (1137) extend longitudinally through partition (1133) above and below central bore (1136), respectively, for permitting passage of pull-wire (W) and conduit (C) therethrough, respectively. In the example shown, an internal bridge (1138) extends laterally across a central region of distal chamber (1134) and a generally cylindrical central bore (1139) extends through bridge (1138) along the longitudinal axis (LA) for permitting passage of working element (101) therethrough, such that bridge (1138) may provide support to working element (101) within distal chamber (1134).

Proximal chamber (1135) of the present example is generally cylindrical and extends longitudinally between partition (1133) and an internal shoulder (1140) of hub (1126). In the example shown, a pair of transversely-opposed alignment slits (1141) extend upwardly and downwardly, respectively, from proximal chamber (1135) along the length thereof. As described in greater detail below, proximal chamber (1135) and slits (1141) are configured to slidably receive respective portions of lead nut (1123) for permitting translation of lead nut (1123) relative to handle body (1112) while inhibiting rotation of lead nut (1123) relative to handle body (1112).

Handle body (1112) of the present example also includes a generally cylindrical proximal bore (1142) extending through collar (1129) along the longitudinal axis (LA) to proximal chamber (1135) and configured to rotatably receive at least a portion of leadscrew (1124). Collar (1129) of handle body (1112) includes a radially outwardly-facing bearing surface (1143) configured to be rotatably received by each of deflection control knob (1122) and strain relief (1125) for facilitating relative rotation between handle body (1112) and each of deflection control knob (1122) and strain relief (1125). In the example shown, a pair of laterally-opposed, distal arcuate slots (1144) extend radially inwardly from bearing surface (1143) to proximal bore (1142) for permitting passage of one or more pins (not shown) therethrough to couple deflection control knob (1122) to leadscrew (1124) and/or for guiding such pins during the relative rotation between handle body (1112) and deflection control knob (1122) to thereby limit such relative rotation between handle body (1112) and deflection control knob (1122). Similarly, a pair of laterally-opposed, proximal arcuate slots (1145) extend radially inwardly from bearing surface (1143) to proximal bore (1142) for guiding corresponding portions of strain relief (1125) during the relative rotation between handle body (1112) and strain relief (1125), as described in greater detail below. While proximal slots (1145) are generally angularly aligned with distal slots (1144) about the longitudinal axis (LA) in the example shown, it will be appreciated that proximal slots (1145) may alternatively be angularly offset from distal slots (1144) about the longitudinal axis (LA). For example, proximal slots (1145) may instead by transversely opposed from each other while distal slots (1144) remain laterally opposed from each other, or distal slots (1144) may instead be transversely opposed from each other while proximal slots (1145) remain laterally opposed from each other.

As shown, a generally annular groove (1146) extends distally from proximal surface (1127) of hub (1126) for receiving a lubricating member in the form of a first O-ring (1147). In this regard, first O-ring (1147) may be formed of a material having a substantially low coefficient of friction, such as polytetrafluoroethylene (PTFE), for reducing friction between hub (1126) and deflection control knob (1122) during relative rotation between handle body (1112) and deflection control knob (1122).

Deflection control knob (1122) of the present example is rotatable relative to handle body (1112) and includes a thumbwheel (1148) and a plurality of grip-enhancing features in the form of protrusions (1149) extending radially outwardly from thumbwheel (1148). Thumbwheel (1148) is positioned such that an operator may rotate thumbwheel (1148) relative to handle body (1112) using the thumb of the hand that is grasping handle body (1112).

Deflection control knob (1122) of the present example also includes a generally cylindrical central bore (1150) extending through thumbwheel (1148) along the longitudinal axis (LA) and configured to rotatably receive collar (1129) of handle body (1112) and, more particularly, a distal portion of bearing surface (1143), for facilitating relative rotation between handle body (1112) and deflection control knob (1122). In the example shown, a pair of laterally-opposed pinholes (1151) extend radially outwardly from central bore (1150) through thumbwheel (1148) for receiving respective pins (not shown) to couple deflection control knob (1122) to leadscrew (1124). When the distal portion of bearing surface (1143) of collar (1129) is rotatably received by central bore (1150), pinholes (1151) may be at a same longitudinal position as distal slots (1144) of handle body (1112) along the longitudinal axis (LA) and may each be generally angularly aligned with at least a portion of a corresponding distal slot (1144) about the longitudinal axis (LA), such that the pins may each extend from the respective pinhole (1151) through the corresponding distal slot (1144) to leadscrew (1124). Due to the arcuate configurations of distal slots (1144), the pins may each be slidable along the corresponding distal slot (1144) and guided thereby during the relative rotation between handle body (1112) and deflection control knob (1122) to define minimum and maximum orientations of deflection control knob (1122) relative to handle body (1112) about the longitudinal axis (LA).

As shown, a distal generally annular groove (1152) extends proximally from a distal surface of thumbwheel (1148) for receiving first O-ring (1147), and a proximal generally annular groove (1153) extends distally from a proximal surface of thumbwheel (1148) for receiving another lubricating member in the form of a second O-ring (1154). In this regard, second O-ring (1154) may be formed of a material having a substantially low coefficient of friction, such as polytetrafluoroethylene (PTFE), for reducing friction between deflection control knob (1122) and strain relief (1125) during relative rotation between deflection control knob (1122) and strain relief (1125). In some versions, deflection control knob (1122) and strain relief (1125) may be spaced apart from each other along the longitudinal axis (LA) by a clearance gap (not shown), such that proximal groove (1153) and/or second O-ring (1154) may be omitted.

Leadscrew (1124) of the present example is rotatable relative to handle body (1112) together with deflection control knob (1122) and includes a generally cylindrical barrel (1155) and a threaded input shaft (1156) extending distally from barrel (1155). In the example shown, barrel (1155) and input shaft (1156) are integrally formed together with each other as a unitary (e.g., monolithic) piece to define leadscrew (1124). In this regard, leadscrew (1124) may be manufactured via 3D printing, injection molding, investment casting, machining, and/or any other suitable manufacturing techniques.

Barrel (1155) of leadscrew (1124) includes a radially outwardly-facing bearing surface (1157) configured to be rotatably received within proximal bore (1142) for facilitating relative rotation between handle body (1112) and leadscrew (1124). A generally frustoconical bore (1158) extends distally and tapers radially inwardly from a proximal surface of barrel (1155), and a generally cylindrical central bore (1159) extends distally from frustoconical bore (1158) through the remainder of barrel (1155) and through input shaft (1156) along the longitudinal axis (LA) for permitting passage of working element (101) therethrough toward distal chamber (1134). In the example shown, a pair of laterally-opposed pinholes (1160) extend radially inwardly from bearing surface (1157) toward central bore (1159) for receiving respective pins (not shown) to couple deflection control knob (1122) to leadscrew (1124). When bearing surface (1157) is rotatably received by central bore (1142) of deflection control knob (1122), pinholes (1160) may be at a same longitudinal position as distal slots (1144) along the longitudinal axis (LA) and may each be generally angularly aligned with at least a portion of a corresponding distal slot (1144) about the longitudinal axis (LA), such that the pins may each extend from the respective pinhole (1160) through the corresponding distal slot (1144) to the corresponding pinhole (1151) of deflection control knob (1122). In this manner, leadscrew (1124) of the present example is non-rotatable (e.g., fixed against rotation) relative to deflection control knob (1122) and is thus rotatable together with deflection control knob (1122) relative to handle body (1112). Such coupling of leadscrew (1124) with deflection control knob (1122) via pins extending through distal slots (1144) may also rotatably capture collar (1129) between bore (1150) of deflection control knob (1122) and bearing surface (1157) of leadscrew (1124), thereby rotatably coupling both leadscrew (1124) and deflection control knob (1122) with handle body (1112).

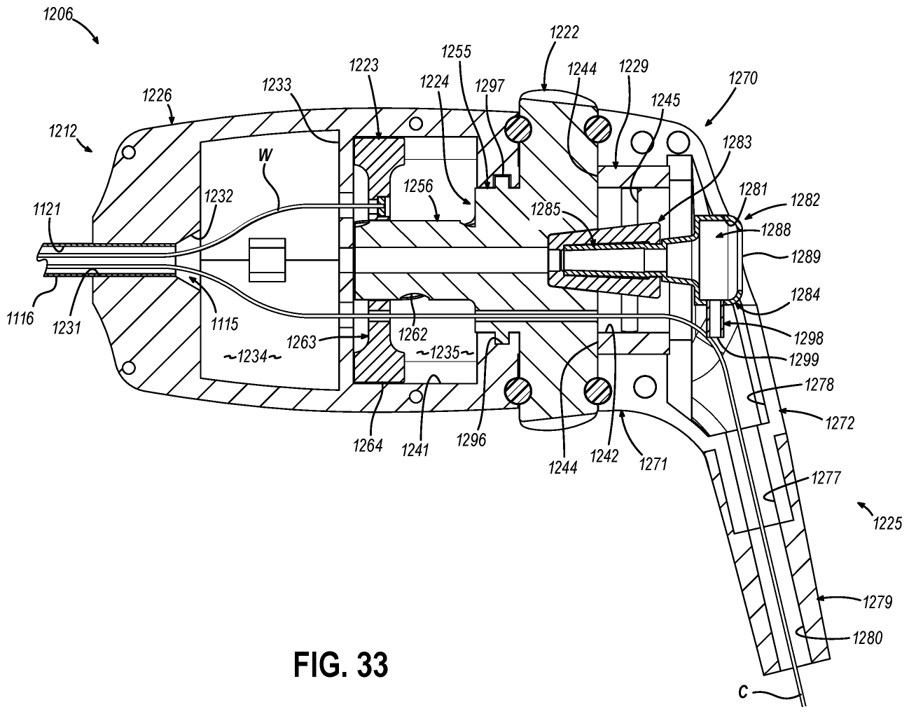

While leadscrew (1124) and deflection control knob (1122) of the present example are individually formed separately from each other as discrete parts and coupled to each other via pins, it will be appreciated that leadscrew (1124) and deflection control knob (1122) may in some cases be integrally formed together with each other as a unitary (e.g., monolithic) piece, such as in a manner similar to that described below in connection with FIG. 33.

In the example shown, a pair of transversely-opposed, arcuate slots (1161) extend longitudinally through barrel (1155) above and below central bore (1159), respectively, for permitting passage of one or more conduits (C) therethrough. Due to the arcuate configurations of slots (1161), the one or more conduits (C) may be routed through the respective slot(s) (1161) when leadscrew (1124) is at a variety of orientations relative to handle body (1112) about the longitudinal axis (LA) and thus may remain routed through the respective slot(s) (1161) during the relative rotation between handle body (1112) and leadscrew (1124) together with deflection control knob (1122). For example, the one or more conduits (C) may be routed through the respective slot(s) (1161) during rotation of leadscrew (1124) with deflection control knob (1122) between the minimum and maximum orientations of deflection control knob (1122) relative to handle body (1112) about the longitudinal axis (LA) while minimizing or avoiding any bending or kinking of the one or more conduits (C) during such rotation and/or any interference of the one or more conduits (C) with such rotation. As shown, arcuate slots (1161) may be angularly offset from pinholes (1160) about the longitudinal axis (LA) and thereby isolated from pinholes (1160), such that the pins extending through pinholes (1160) may not present a snag hazard for the one or more conduits (C). In the example shown, a plurality of (e.g., three) threads in the form of helical flutes (1162) extend radially inwardly from a generally cylindrical outer surface of input shaft (1156) along the length thereof for threadably engaging corresponding portions of lead nut (1123).

Lead nut (1123) of the present example is non-rotatable (e.g., fixed against rotation) relative to handle body (1112) of handle assembly (1106), and includes a ring (1163) and a pair of transversely-opposed alignment tabs or rails (1164) extending upwardly and downwardly, respectively, from a generally cylindrical outer surface of ring (1163). Ring (1163) is sized and shaped to be slidably received within proximal chamber (1135) of handle body (1112), and alignment rails (1164) are sized and shaped to be slidably received within corresponding slits (1141) of handle body (1112) to facilitate translation of lead nut (1123) relative to handle body (1112) along the longitudinal axis (LA) between the distal position shown in FIG. 32A and the proximal position shown in FIG. 32B, while inhibiting rotation of lead nut (1123) relative to handle body (1112) about the longitudinal axis (LA). In this regard, lead nut (1123) of the present example also includes a central bore (1165) extending through ring (1163) along the longitudinal axis (LA) and configured to receive input shaft (1156) of leadscrew (1124), as described in greater detail below.

In the example shown, a pull-wire grounding member in the form of a multistage bore (1166) extends longitudinally through ring (1163) above central bore (1165) for anchoring pull-wire (W) to lead nut (1123) via a grommet (1167) secured thereto. In this manner, the proximal end of pull-wire (W) is fixedly secured to lead nut (1123) of the present example and is thus translatable together with lead nut (1123) relative to handle body (1112).

In the example shown, at least one arcuate slot (1168) extends longitudinally through ring (1163) below central bore (1165) (e.g., transversely opposite multistage bore (1166)) for permitting passage of one or more conduits (C) therethrough. Arcuate slot (1168) of the present example is at least partially aligned with lower elongate slot (1137) of partition (1133) such that the one or more conduit (C) may be routed proximally from lower elongate slot (1137) through slot (1168) toward a corresponding slot (1161) of leadscrew (1124). Due to the arcuate configuration of slot (1168), at least a portion of slot (1168) may be aligned with at least a portion of a corresponding slot (1161) of leadscrew (1124) when leadscrew (1124) is at a variety of orientations relative to handle body (1112) about the longitudinal axis (LA) and thus may remain at least partially aligned with the respective slot (1161) during the relative rotation between handle body (1112) and leadscrew (1124) together with deflection control knob (1122). Due to the at least partial alignment of slot (1168) with each of lower elongate slot (1137) of partition (1133) and a corresponding slot (1161) of leadscrew (1124), the one or more conduits (C) may be routed through slot (1168) when lead nut (1123) is at a variety of longitudinal positions relative to handle body (1112) along the longitudinal axis (LA) and thus may remain routed through slot (1168) during the translation of lead nut (1123) relative to handle body (1112). For example, the one or more conduits (C) may be routed through slot (1168) during translation of lead nut (1123) between the distal and proximal positions while minimizing or avoiding any bending or kinking of the one or more conduits (C) during such translation and/or any interference of the one or more conduits (C) with such translation.

Lead nut (1123) of the present example further includes a plurality of (e.g., three) threads in the form of ridges (1169) protruding radially inwardly from central bore (1165) for threadably engaging corresponding flutes (1162) of leadscrew (1124). Each ridge (1169) may extend along a helical path similar to that of the corresponding flute (1162) for promoting the threadable engagement between each ridge (1169) and the corresponding flute (1162). It will be appreciated that the threads of lead nut (1123) and leadscrew (1124) may be of any suitable form for threadably engaging each other, and are not limited to the particular configurations of ridges (1169) and flutes (1162) shown. In any event, the threadable engagement between ridges (1169) and the corresponding flutes (1162) may facilitate conversion of the rotary movement of leadscrew (1124) about the longitudinal axis (LA) into linear movement of lead nut (1123) along the longitudinal axis (LA), such that rotation of leadscrew (1124) (e.g., via rotation of deflection control knob (1122)) may drive translation of lead nut (1123) between the distal position shown in FIG. 32A and the proximal position shown in FIG. 32B. Thus, lead nut (1123) and leadscrew (1124) may collectively define a screw-driven linear actuator. In some versions, the distal position of lead nut (1123) shown in FIG. 32A may correspond to one of the minimum or maximum orientations of deflection control knob (1122) relative to handle body (1112) about the longitudinal axis (LA), and the proximal position of lead nut (1123) shown in FIG. 32B may correspond to the other of the minimum or maximum orientations of deflection control knob (1122) relative to handle body (1112) about the longitudinal axis (LA).

As noted above, the proximal end of pull-wire (W) is secured to lead nut (1123), such that pull-wire (W) translates with lead nut (1123) relative to handle body (1112) in response to rotation of deflection control knob (1122) relative to handle body (1112). As also noted above, proximal retraction of pull-wire (W) relative to handle body (1112) causes bending of flexible portion (1118) and thus deflection of distal end (1120) laterally away from the longitudinal axis (LA) of rigid portion (1116). In some versions, flexible portion (1118) may be configured to resiliently assume a straight configuration when lead nut (1123) is in the distal position, such that distal end (1120) may resiliently realign with the longitudinal axis (LA) of rigid portion (1116) when lead nut (1123) is returned from the proximal position to the distal position. In some other versions, pull-wire (W) may have sufficient column strength to urge flexible portion (1118) to a straight configuration and thereby realign distal end (1120) with the longitudinal axis (LA) of rigid portion (1116) when lead nut (1123) is returned from the proximal position to the distal position. In such cases, pull-wire (W) may be referred to as a push-pull wire.

In some versions, the helical path along which each flute (1162) of leadscrew (1124) extends may be configured to facilitate lateral deflection of distal end (1120) from about 0° relative to the longitudinal axis (LA) to about 110° relative to the longitudinal axis (LA) in response to rotation of deflection control knob (1122) together with leadscrew (1124) through a range of motion of less than or equal to about 45°. In addition, or alternatively, the helical path along which each flute (1162) of leadscrew (1124) extends may vary to provide a variable conversion of rotary movement into linear movement. For example, such varying helical paths may allow rotation of leadscrew (1124) through a first range of motion to drive translation of lead nut (1123) a first distance and may allow rotation of leadscrew (1124) through a second range of motion having a same magnitude as the first range of motion to drive translation of lead nut (1123) a second distance different from the first distance.

While the linear output member of the linear actuator has been described in the form of lead nut (1123) and the rotary input member of the linear actuator has been described in the form of leadscrew (1124), it will be appreciated that the linear output and rotary input members of the linear actuator may be of any suitable form for converting a rotary input into a linear output. For example, the rotary input member may be provided in the form of a lead nut and the rotary output member may be provided in the form of a leadscrew. Also, while a screw-driven linear actuator has been described as being collectively defined by lead nut (1123) and leadscrew (1124), it will be appreciated that any other suitable type of linear actuator, such as a cam-driven linear actuator, may be used to convert rotation of deflection control knob (1122) into translation of pull-wire (W), such that pull-wire (W) translates relative to handle body (1112) in response to rotation of deflection control knob (1122) relative to handle body (1112).

Strain relief (1125) of the present example is rotatable relative to handle body (1112) independently of leadscrew (1124) and deflection control knob (1122), and includes a rigid strain relief body (1170) having a generally annular cuff (1171) and a generally tubular trunk (1172) extending both proximally and transversely outwardly from cuff (1171). In the example shown, strain relief body (1170) is formed from first and second shells (e.g., halves) (1170a, 1170b) fixedly coupled to each other along a longitudinal centerline of strain relief body (1170) via corresponding pairs of engagement features in the form of detents (1173a) and indents (1173b), such that shells (1170a, 1170b) collectively define strain relief body (1170). Shells (1170a, 1170b) may each be manufactured via 3D printing, injection molding, investment casting, machining, and/or any other suitable manufacturing techniques, and subsequently assembled to each other to form strain relief body (1170). It will be appreciated that strain relief body (1170) may be formed in any other suitable manner. For example, shells (1170a, 1170b) may be integrally formed together with each other as a unitary (e.g., monolithic) piece to define strain relief body (1170), such that detents (1173a) and indents (1173b) may be omitted.

Strain relief (1125) of the present example also includes a generally cylindrical central bore (1174) extending through cuff (1171) along the longitudinal axis (LA) and configured to rotatably receive collar (1129) of handle body (1112) and, more particularly, a proximal portion of bearing surface (1143), for facilitating relative rotation between handle body (1112) and strain relief (1125). In the example shown, a pair of laterally-opposed detents (1175) (one shown) extend radially inwardly from central bore (1174) for receipt within respective proximal slots (1145) of handle body (1112) to rotatably couple strain relief (1125) to handle body (1112). When the proximal portion of bearing surface (1143) of collar (1129) is rotatably received by central bore (1174), detents (1175) may be at a same longitudinal position as proximal slots (1145) along the longitudinal axis (LA) and may each be generally angularly aligned with at least a portion of a corresponding proximal slot (1145) about the longitudinal axis (LA), such that detents (1175) may each extend at least partially through the corresponding proximal slot (1145). Due to the arcuate configurations of proximal slots (1145), detents (1175) may each be slidable along the corresponding proximal slot (1145) and guided thereby during the relative rotation between handle body (1112) and strain relief (1125) to define minimum and maximum orientations of strain relief (1125) relative to handle body (1112) about the longitudinal axis (LA).

In some versions, detents (1175) may each include a radially inner head to rotatably capture collar (1129) between central bore (1174) of strain relief (1125) and such radially inner heads. Such inner heads may each be sized to fit through one or more corresponding enlarged ends of proximal slots (1145) for facilitating insertion and removal of detents (1175) into and out of proximal slots (1145) when strain relief (1125) is at the minimum and/or maximum orientations relative to handle body (1112), to thereby provide a bayonet-style coupling between strain relief (1125) and handle body (1112).

As shown, a generally annular groove (1176) extends proximally from the distal surface of cuff (1171) for receiving second O-ring (1154). As noted above, deflection control knob (1122) and strain relief (1125) may be spaced apart from each other along the longitudinal axis (LA) by a clearance gap (not shown), such that groove (1176) and/or second O-ring (1154) may be omitted. In such cases, a generally annular groove may extend radially outwardly from central bore (1174) of strain relief (1125) and another generally annular groove may extend radially inwardly from bearing surface (1143) of collar (1129) for collectively receiving an O-ring.

Strain relief (1125) of the present example also includes a generally cylindrical outlet (1177) extending through trunk (1172) along an exit axis (EA) that is oriented obliquely relative to the longitudinal axis (LA). In the example shown, outlet (1177) is in communication with central bore (1174) via a curved and/or angled interior cavity (1178) which transitions from extending along the longitudinal axis (LA) near central bore (1174) to extending along the exit axis (EA) near outlet (1177), such that conduit (C) may extend proximally and/or transversely outwardly through cavity (1178) to outlet (1177) from proximal bore (1142) of handle body (1112) when the proximal portion of bearing surface (1143) of collar (1129) is rotatably received by central bore (1174). In this manner, outlet (1177) of strain relief (1125) is configured to direct the proximal portion of conduit (C) away from the longitudinal axis (LA) along a path that extends proximally from handle assembly (1106) along exit axis (EA).

Strain relief (1125) of the present example also includes a flexible strain relief extension (1179) coupled to trunk (1172) of strain relief body (1170), and a generally cylindrical outlet extension (1180) extending through strain relief extension (1179) coaxially with outlet (1177) for directing the proximal portion of conduit (C) proximally from outlet (1177) of trunk (1172) further away from the longitudinal axis (LA) along the exit axis (EA). In the example shown, flexible strain relief extension (1179) at least partially encases a proximal end of trunk (1172) of strain relief body (1170) and may thereby assist with coupling shells (1170a, 1170b) to each other.

In some versions, proximal slots (1145) may permit rotation of strain relief (1125) between the minimum and maximum orientations relative to handle body (1112) about the longitudinal axis (LA) through a range of motion of at least about 180° and may thereby permit rotation of the exit axis (EA) (and the proximal portion of conduit (C) extending therealong) between the same minimum and maximum orientations relative to handle body (1112) about the longitudinal axis (LA) through the same range of motion.

In the example shown, a proximal aperture (1181) extends through trunk (1172) of strain relief (1125) along the longitudinal axis (LA) for facilitating insertion of working element (101) and/or one or more suction components therethrough. In this regard, handle assembly (1106) of the present example further includes a suction valve (1182) rotatably mounted to leadscrew (1124) via an adapter (1183) such that suction valve (1182) may be rotatable relative to handle body (1112), independently of leadscrew (1124), about the longitudinal axis (LA) to reorient a proximal suction tube (not shown) proximally exiting handle assembly (1106) relative to the longitudinal axis (LA) while providing suction to a distal suction tube (not shown) via the proximal suction tube (not shown). Such a proximal suction tube may extend proximally from handle assembly (1106) to a source of suction (not shown), and such a distal suction tube may extend distally through central bore (1159) of leadscrew (1124), central bore (1136) of partition (1133), central bore (1139) of bridge (1138), frustoconical bore (1132) of hub (1126), and working lumen (1121) of shaft assembly (1108) to open distal end (1120), such as for placing working channel (154) of distal endoscope cap (110) in fluid communication with the source of suction to thereby draw excess liquids and debris away from cameras (164) of distal endoscope cap (110).

Suction valve (1182) of the present example is rotatable relative to handle body (1112) independently of leadscrew (1124) and deflection control knob (1122), and includes a generally mushroom-shaped head (1184) and a generally cylindrical shank (1185) extending distally from head (1184). Shank (1185) of suction valve (1182) includes a radially outwardly-facing bearing surface (1186) configured to be rotatably received by adapter (1183) for facilitating relative rotation between shank (1185) and adapter (1183). In the example shown, a generally annular recess (1187) extends radially inwardly from bearing surface (1186) for rotatably coupling suction valve (1182) to adapter (1183).

Suction valve (1182) of the present example also includes a suction lumen (1188) extending through both head (1184) and shank (1185) along the longitudinal axis (LA) and including a proximal inlet port (1189). Inlet port (1189) may be configured to receive a distal end and/or a distal fluid coupling of the proximal suction tube, to provide a fluid-tight connection between suction lumen (1188) and a lumen of the proximal suction tube, such that suction lumen (1188) may be in fluid communication with the source of suction via the proximal suction tube. In addition or alternatively, inlet port (1189) may be configured to facilitate insertion of working element (101) through suction lumen (1188) and into a lumen of the distal suction tube toward open distal end (1120).

Adapter (1183) of the present example is rotatable relative to handle body (1112) together with leadscrew (1124) and deflection control knob (1122), and includes a generally frustoconical sleeve bushing (1190). Sleeve bushing (1190) of adapter (1183) includes a radially outwardly-facing grip surface (1191) configured to be fixedly received within frustoconical bore (1158) for fixedly securing adapter (1183) to leadscrew (1124). For example, grip surface (1191) may be tapered radially inwardly in a distal direction for providing a press-fit engagement between grip surface (1191) and frustoconical bore (1158).

Adapter (1183) of the present example also includes a proximal socket (1192) extending distally from a proximal surface of sleeve bushing (1190) and configured to rotatably receive shank (1185) of suction valve (1182) and, more particularly, bearing surface (1186), and a distal socket (1193) extending proximally from a distal surface of sleeve bushing (1190) and configured to rotatably receive a proximal end and/or a proximal fluid coupling of the distal suction tube, to provide a fluid-tight connection between suction lumen (1188) and a lumen of the distal suction tube, such that the lumen of the distal suction tube may be in fluid communication with the source of suction via suction valve (1182) and the proximal suction tube while facilitating relative rotation between suction valve (1182) and the distal suction tube about the longitudinal axis (LA). In the example shown, a proximal generally annular flange (1194) extends radially inwardly from proximal socket (1192) for receipt within recess (1187) of suction valve (1182) to rotatably couple suction valve (1182) to adapter (1183), and a distal generally annular flange (1195) extends radially inwardly from distal socket (1193) to rotatably couple the proximal end and/or proximal fluid coupling of the distal suction tube to adapter (1183), and/or to define a seal for providing the fluid-tight connection between suction lumen (1188) and the lumen of the distal suction tube.

While adapter (1183) has been described as being fixedly secured to leadscrew (1124) and rotatable therewith relative to handle body (1112) about the longitudinal axis (LA), adapter (1183) may in some other versions be fixedly secured to the distal suction tube and rotatable therewith relative to each of leadscrew (1124) and suction valve (1182) about the longitudinal axis (LA), and may in some other versions be fixedly secured to suction valve (1182) and rotatable therewith relative to each of leadscrew (1124) and the distal suction tube about the longitudinal axis (LA). In any event, suction valve (1182) is rotatable relative to the distal suction tube about the longitudinal axis (LA), such that suction valve (1182) may rotate relative to handle body (1112) about the longitudinal axis (LA) while the distal suction tube remains stationary relative to handle body (1112) and capable of receiving suction and/or working element (101).

In some versions, adapter (1183) may permit rotation of suction valve (1182) between minimum and maximum orientations relative to the distal suction tube about the longitudinal axis (LA) through a range of motion of at least about 180° and may thereby permit rotation of the proximal suction tube between the same minimum and maximum orientations relative to the distal suction tube about the longitudinal axis (LA) through the same range of motion. In some cases, suction valve (1182) may be non-rotatable (e.g., fixed against rotation) relative to strain relief (1125) and may thus be rotatable together with strain relief (1125) relative to handle body (1112). While suction valve (1182) of the present example includes a single inlet port (1189) extending along the longitudinal axis (LA) for receiving both working element (101) and the proximal suction tube, it will be appreciated that suction valve (1182) may in some cases include a second inlet port extending along a different axis (e.g., exit axis (EA) or an axis that is oriented generally perpendicularly relative to the longitudinal axis (LA)) for receiving the proximal suction tube, such as in a manner similar to that described below in connection with FIG. 33.

### B. Example of a Handle Assembly Having Deflection Control Knob Integrally Formed with Leadscrew for Actuating Deflection of Distal Shaft End

In some instances, it may be desirable to provide a handle assembly having a deflection control knob and a leadscrew that are integrally formed together with each other as a unitary (e.g., monolithic) piece to facilitate or otherwise improve manufacturability of the handle assembly, such as by reducing the quantity of separate pieces that collectively form the handle assembly. In addition, or alternatively, it may be desirable to provide a handle assembly having a suction valve having separate inlet ports respectively receiving working element (101) and the proximal suction tube, such as for enabling the proximal suction tube to proximally exit the handle assembly generally coaxially with the conduit (C) (e.g., along the exit axis (EA)). FIG. 33 shows an example of a handle assembly (1206) having such functionalities, and which may be incorporated into instrument (1100) in place of handle assembly (1106). Handle assembly (1206) may be similar to handle assembly (1106) described above except as otherwise described below. In this regard, handle assembly (1206) of the present example includes a handle body (1212), a deflection control knob (1222), a lead nut (1223), a leadscrew (1224), and a strain relief (1225), which are similar to handle body (1112), deflection control knob (1122), lead nut (1123), leadscrew (1124), and strain relief (1125) described above, respectively, except as otherwise described below.

Handle body (1212) of the present example includes a hub (1226), a collar (1229), a distal bore (1231), a frustoconical bore (1232), a partition (1233) defining a distal chamber (1234) and a proximal chamber (1235), a pair of slits (1241), a proximal bore (1242), a pair of distal arcuate slots (1244), and a pair of proximal arcuate slots (1245) (one shown). In the example shown, distal slots (1244) are transversely opposed from each other while proximal slots (1245) are laterally opposed from each other, such that proximal slots (1245) are angularly offset from distal slots (1244) about the longitudinal axis of rigid portion (1116) of shaft assembly (1108). Handle body (1212) of the present example also includes a generally annular recess (1296) extending radially outwardly from proximal bore (1242) for rotatably coupling deflection control knob (1222) and leadscrew (1224) to handle body (1212).

In the example shown, deflection control knob (1222) and leadscrew (1224) are integrally formed together with each other as a unitary (e.g., monolithic) piece. In this regard, leadscrew (1224) of the present example includes a barrel (1255), a threaded input shaft (1256), and a plurality of flutes (1262), and deflection control knob (1222) extends radially outwardly from a bearing surface of barrel (1255) through distal slots (1244) of handle body (1212). Leadscrew (1224) of the present example also includes a generally annular flange (1297) extending radially outwardly from the bearing surface of barrel (1255) for receipt within recess (1296) of handle body (1212) to rotatably couple deflection control knob (1222) and leadscrew (1224) to handle body (1212). Lead nut (1223) of the present example includes a ring (1263), a pair of rails (1264), and a plurality of ridges (not shown) configured to threadably engage flutes (1262) in a manner similar to that described above in connection with FIGS. 32A-32B.

Strain relief (1225) of the present example includes a strain relief body (1270) having a cuff (1271) and a trunk (1272), a central bore (1274), a pair of laterally-opposed detents (not shown) for receipt within proximal slots (1245) of handle body (1212), an outlet (1277), a cavity (1278), a strain relief extension (1279), an outlet extension (1280), and a proximal aperture (1281). Outlet (1277) and outlet extension (1280) each extend along an exit axis that is oriented obliquely relative to the longitudinal axis of rigid portion (1116) of shaft assembly (1108) to direct the proximal portion of conduit (C) away from the longitudinal axis of rigid portion (1116) along a path that extends proximally from handle assembly (1206) along the exit axis.

Handle assembly (1206) of the present example further includes a suction valve (1282) rotatably mounted to leadscrew (1224) via an adapter (1283). Suction valve (1282) of the present example includes a head (1284), a shank (1285), and a suction lumen (1288) including a first inlet port (1289). In the example shown, first inlet port (1289) extends along the longitudinal axis of rigid portion (1116) of shaft assembly (1108) for receiving working element (101), and suction valve (1282) further includes a coupling (1298) extending transversely (e.g., downwardly) from head (1284) and defining a second inlet port (1299) in fluid communication with suction lumen (1288). Second inlet port (1299) may be configured to receive a distal end and/or a distal fluid coupling of the proximal suction tube, to provide a fluid-tight connection between suction lumen (1288) and a lumen of the proximal suction tube, such that suction lumen (1288) may be in fluid communication with the source of suction via the proximal suction tube. In the example shown, second inlet port (1299) extends along an axis that is oriented generally perpendicularly relative to the longitudinal axis of rigid portion (1116) of shaft assembly (1108), such that the proximal suction tube may proximally exit handle assembly (1206) generally coaxially with the proximal portion of conduit (C), such as along the exit axis of outlet (1277) and outlet extension (1280). In this manner, the proximal suction tube may be reoriented together with the proximal portion of conduit (C) about the longitudinal axis of rigid portion (1116) via rotation of strain relief (1225) relative to handle body (1212).

### IV. EXAMPLE OF AN IRRIGATION FLUID DISPENSING ASSEMBLY

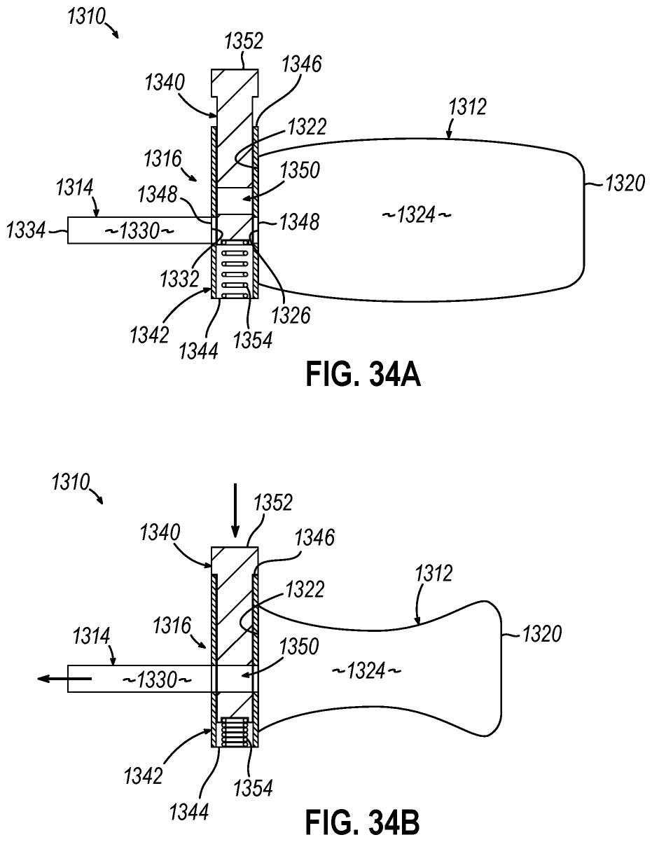

In some instances, it may be desirable to equip an ENT instrument, such as any of ENT instruments (100, 1100), with an irrigation fluid dispensing assembly having an actuator that may be incorporated directly into the ENT instrument to reduce the quantity of peripheral components that support the ENT instrument, such as by reducing or eliminating the ENT instrument's dependence on pumps, foot pedals, wires, and/or inflation devices to dispense irrigation fluid. FIGS. 34A-34B show an example of an irrigation fluid dispensing assembly (1310) having such functionalities, and which may be incorporated into any of instruments (100, 1100), such as into the respective handle assembly (106, 1106, 1206) thereof.

Dispensing assembly (1310) of the present example includes an irrigation fluid reservoir in the form of a flexible bladder (1312), an irrigation fluid tube (1314), and a valve (1316) positioned between bladder (1312) and irrigation fluid tube (1314). In some versions, bladder (1312) may be housed within any of handle assemblies (106, 1106, 1206), such as within any of handle bodies (112, 1112, 1212), and irrigation fluid tube (1314) may extend distally therefrom, such as to inlet port (172) of distal endoscope cap (110).

Bladder (1312) of the present example extends between proximal and distal ends (1320, 1322) and has a collapsible interior chamber (1324) configured to contain irrigation fluid (e.g., saline or any other suitable liquid). In the example shown, a discharge orifice (1326) extends through distal end (1322) of bladder (1312) and is in fluid communication with chamber (1324) for facilitating loading of irrigation fluid into chamber (1324) and dispensing of irrigation fluid from chamber (1324). In this regard, bladder (1312) of the present example is expandable and contractable between at least one expanded state (FIG. 34A) and a contracted state (FIG. 34B) and is resiliently biased toward the contracted state, such that chamber (1324) may initially be collapsed prior to loading of chamber (1324) with irrigation fluid and may subsequently be inflated with irrigation fluid to transition bladder (1312) toward the at least one expanded state. In this manner, chamber (1324) may be pressurized with irrigation fluid when bladder (1312) is in the at least one expanded state. Bladder (1312) may be formed of any suitable material, such as an elastomeric material. In some versions, bladder (1312) may have an accordion configuration. In any event, a luer attachment (not shown) may be coupled to discharge orifice (1326) to facilitate loading of irrigation fluid into chamber (1324).

Irrigation fluid tube (1314) of the present example includes an irrigation lumen (1330) having a proximal inlet port (1332) and a distal outlet port (1334). In the example shown, proximal inlet port (1332) is axially aligned with discharge orifice (1326) of bladder (1312) and is configured to be selectively placed into and out of fluid communication therewith via valve (1316), as described in greater detail below. Distal outlet port (1334) may be configured to be received by inlet port (172) of distal endoscope cap (110) to provide a fluid-tight connection between irrigation lumen (170) of distal endoscope cap (110) and irrigation lumen (1330) for selectively placing irritation lumen (170) in fluid communication with chamber (1324) of bladder (1312).

Valve (1316) of the present example includes a valve stem (1340) slidably housed within a sleeve (1342) such that valve stem (1340) is configured to translate relative to sleeve (1342) between an upper, unactuated position (FIG. 34A) and a lower, actuated position (FIG. 34B). In the example shown, sleeve (1342) extends between a closed lower end (1344) and an open upper end (1346) configured to slidably receive valve stem (1340). A pair of axially-aligned openings (1348) extend through opposing sides of sleeve (1342) and are in fluid communication with discharge orifice (1326) of bladder (1312) and proximal inlet port (1332) of irrigation fluid tube (1314), respectively, and are configured to be selectively placed into and out of fluid communication with each other via valve stem (1340). In this regard, a passageway (1350) extends through valve stem (1340) in a direction perpendicular to a length of valve stem (1340). Passageway (1350) of the present version is configured to be axially offset from each of openings (1348) (and thus from each of discharge orifice (1326) and proximal inlet port (1332)) when valve stem (1340) is in the unactuated position shown in FIG. 34A such that irrigation lumen (1330) of irrigation fluid tube (1314) is fluidly isolated from chamber (1324) of bladder (1312), and is configured to be at least partially axially aligned with each of openings (1348) (and thus with each of discharge orifice (1326) and proximal inlet port (1332)) when valve stem (1340) is in the actuated position shown in FIG. 34B such that irrigation lumen (1330) of irrigation fluid tube (1314) is in fluid communication with chamber (1324) of bladder (1312).

In the example shown, valve stem (1340) protrudes upwardly from open end (1346) of sleeve (1342) to present an actuator in the form of a button (1352). In some versions, button (1352) may extend outwardly through a portion of the respective handle body (112, 1112, 1212). Button (1352) of the present example is configured to be depressed downwardly by the operator (e.g., using a single finger or the thumb of the hand that is grasping handle body (112, 1112, 1212)) to transition valve stem (1340) from the unactuated state toward the actuated state, as shown in FIG. 34B. Due to the pressurization of chamber (1324) with irrigation fluid, the irrigation fluid may flow from the relatively high fluid pressure of chamber (1324) toward the relatively low fluid pressure of irrigation lumen (1330) via passageway (1350). As shown in FIG. 34B, bladder (1312) may resiliently contract and thereby collapse chamber (1324) as the fluid pressure within chamber (1324) decreases due to the dispensing of irrigation fluid therefrom. In some versions, a one-way valve (not shown) may be positioned at or near proximal inlet port (1332) of irrigation fluid tube (1314) for preventing irrigation fluid from flowing back toward chamber (1324) (e.g., once the fluid pressure within chamber (1324) has decreased to be substantially equal to that within irrigation lumen (1330)).

In the example shown, valve stem (1340) is resiliently biased upwardly toward the unactuated position via a biasing member in the form of a compression spring (1354) positioned between valve stem (1340) and closed end (1344) of sleeve (1342), such that valve stem (1340) may resiliently return to the unactuated state when button (1352) is released by the operator. Such resilient biasing of valve stem (1340) may prevent inadvertent dispensing of irrigation fluid from chamber (1324) and/or assist with regulating the dispensing of irrigation fluid from chamber (1324) in a controlled manner. In some cases, spring (1354) may control the amount (e.g., volume) of irrigation fluid dispensed when button (1352) is depressed by limiting the extent to which button (1352) may be depressed.

While valve (1316) has been described as being positioned between bladder (1312) and irrigation fluid tube (1314), it will be appreciated that valve (1316) may be positioned at any other suitable location for selectively placing at least a portion of chamber (1324) into and out of fluid communication with at least a portion of irrigation lumen (1330). For example, valve (1316) may be positioned within either chamber (1324) or irrigation lumen (1330). While the irrigation fluid reservoir has been described in the form of flexible bladder (1312), it will be appreciated that any other suitable form of reservoir may be used for pressurizing an interior chamber thereof with irrigation fluid. For example, the irrigation fluid reservoir may instead be provided in the form of a rigid housing having an irrigation fluid interior chamber and an air interior chamber separated from each other by a flexible or movable partition for permitting inflation and collapsing of the irrigation fluid interior chamber.

### V. EXAMPLES OF A DISTAL ENDOSCOPE CAP WITH A NAVIGATION SENSOR

As noted above, any of the examples of distal endoscope caps (110, 210, 310, 410, 510, 610, 710) described in connection with FIGS. 3-26 may include a navigation sensor assembly (152) operable to generate signals indicative of the position of the distal endoscope cap (110, 210, 310, 410, 510, 610, 710). As also noted above, such a navigation sensor assembly (152) may be incorporated into any of the examples of distal endoscope caps (110, 210, 310, 410, 510, 610, 710) at any suitable location, such as within the respective bore (150, 250, 350, 450, 550, 651, 750) or along a generally cylindrical outer surface of the respective hub (142, 242, 342, 542, 642, 742). In some instances, it may be desirable to incorporate navigation sensor assembly (152) in an interior space of a distal endoscope cap (110, 210, 310, 410, 510, 610, 710) other than within the respective bore (150, 250, 350, 450, 550, 651, 750). Each of the examples of distal endoscope caps (1410, 1510) described below may function in such a manner.

### A. Example of a Distal Endoscope Cap with Two Cameras, Single Illuminating Element, and Single Navigation Sensor

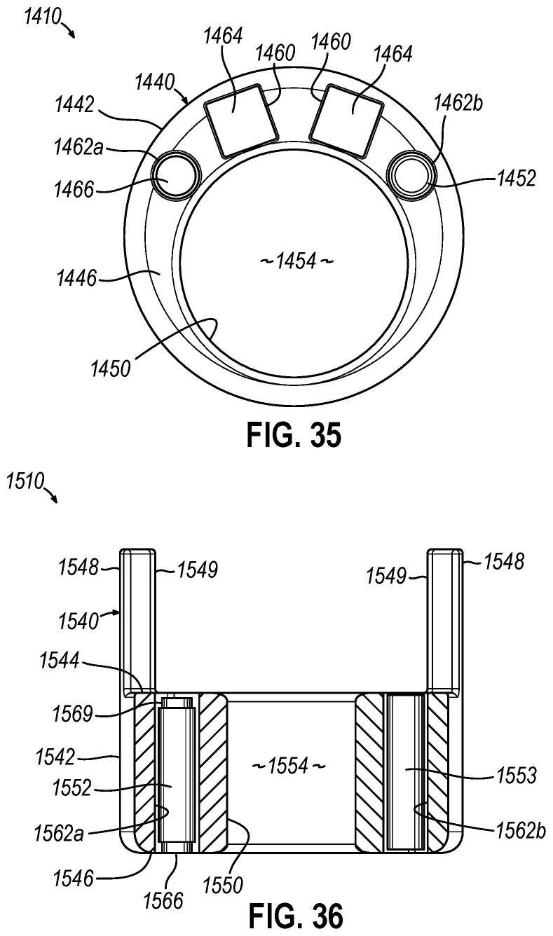

FIG. 35 shows an example of a distal endoscope cap (1410) which may be incorporated into instrument (100) in place of distal endoscope cap (110). Distal endoscope cap (1410) may be similar to distal endoscope cap (110) described above except as otherwise described below. In this regard, distal endoscope cap (1410) of the present example includes a body (1440) having a generally cylindrical hub (1442) extending between a proximal surface (not shown) and a distal surface (1446).

While not shown, body (1440) may have a pair of laterally-opposed coupling wings extending proximally from the proximal surface of hub (1442) and similar to wings (148), for example. Body (1440) may be constructed of any suitable translucent or opaque material, such as a polymeric material (e.g., plastic) or a metallic material. Hub (1442) and the wings may be integrally formed together with each other as a unitary (e.g., monolithic) piece to define body (1440). In this regard, body (1440) may be manufactured via 3D printing, injection molding, investment casting, machining, and/or any other suitable manufacturing techniques.

Each wing of distal endoscope cap (1410) may include a laterally inwardly-facing gripping surface (not shown) configured to frictionally engage a generally cylindrical outer surface of flexible portion (118) of shaft assembly (108) near open distal end (120) for attaching distal endoscope cap (1410) to open distal end (120) in a manner similar to that described above. Alternatively, distal endoscope cap (1410) may be secured at open distal end (120) using any other suitable structure or techniques.

Distal endoscope cap (1410) of the present example also includes a generally cylindrical bore (1450) extending longitudinally between the proximal surface and distal surface (1446) of hub (1442) and configured to be axially aligned with the working lumen of shaft assembly (108) when distal endoscope cap (1410) is attached to open distal end (120), such that working element (101) may pass through bore (1450) as working element (101) is guided through distal open end (120). Thus, a working channel (1454) may extend along bore (1450).

Distal endoscope cap (1410) of the present example also includes an arch-shaped array of generally rectangular and generally cylindrical bores (1460, 1462a, 1462b) each extending longitudinally between the proximal surface and distal surface (1446) of hub (1442) and disposed about (e.g., above) bore (1450). More particularly, distal endoscope cap (1410) includes an inner pair of laterally-opposed, generally rectangular bores (1460) and an outer pair of laterally-opposed, generally cylindrical bores (1462a, 1462b). Distal endoscope cap (1410) further includes a pair of cameras (1464) similar to cameras (164), which are each received within a respective inner bore (1460) in a manner similar to that described above; and a single illuminating element (1466) similar to illuminating elements (166), which is received within first outer bore (1462a) in a manner similar to that described above. In some versions, illuminating element (1466) may include fiber optic components, such as a lens that is optically coupled with one or more respective optical fibers or optical fiber bundles.

As shown, distal endoscope cap (1410) of the present example also includes a single navigation sensor (1452), which is received within second outer bore (1462b). In some versions, navigation sensor (1452) may be configured as a single-axis sensor. In addition, or alternatively, navigation sensor (1452) may be similar to navigation sensor assembly (152). In some cases, a second illuminating element (1466) may also be received within second outer bore (1462b) and may be provided in the form of a lens that is optically coupled with one or more respective optical fibers or optical fiber bundles. In some such cases, navigation sensor (1452) may be disposed (e.g., wrapped) about the optical fiber(s) or optical fiber bundle(s) of the second illuminating element (1466).

While not shown, distal endoscope cap (1410) of the present example may also include an irrigation lumen extending distally from the proximal surface toward distal surface (1446) of hub (1442) and disposed above bore (1450) between inner bores (1460) for directing streams of liquid generally laterally outwardly into inner bores (1460) through fluid outlet slots (not shown) in a manner similar to that described above.

### B. Example of a Distal Endoscope Cap with Two Cameras, Single Illuminating Element, Single Navigation Sensor, and Single Optical Sensor

FIG. 36 shows another example of a distal endoscope cap (1510) which may be incorporated into instrument (100) in place of distal endoscope cap (110). Distal endoscope cap (1510) may be similar to distal endoscope cap (110) described above except as otherwise described below. In this regard, distal endoscope cap (1510) of the present example includes a body (1540) having a generally cylindrical hub (1542) extending between proximal and distal surfaces (1544, 1546).

As shown, body (1540) has a pair of laterally-opposed coupling wings (1548) extending proximally from proximal surface (1544) of hub (1542). Body (1540) may be constructed of any suitable translucent or opaque material, such as a polymeric material (e.g., plastic) or a metallic material. In the example shown, hub (1542) and wings (1548) are integrally formed together with each other as a unitary (e.g., monolithic) piece to define body (1540). In this regard, body (1540) may be manufactured via 3D printing, injection molding, investment casting, machining, and/or any other suitable manufacturing techniques.

In the example shown, each wing (1548) of distal endoscope cap (1510) includes a laterally inwardly-facing gripping surface (1549) configured to frictionally engage a generally cylindrical outer surface of flexible portion (118) of shaft assembly (108) near open distal end (120) for attaching distal endoscope cap (1510) to open distal end (120) in a manner similar to that described above. Alternatively, distal endoscope cap (1510) may be secured at open distal end (120) using any other suitable structure or techniques.

Distal endoscope cap (1510) of the present example also includes a generally cylindrical bore (1550) extending longitudinally between proximal and distal surfaces (1544, 1546) of hub (1542) and configured to be axially aligned with the working lumen of shaft assembly (108) when distal endoscope cap (1510) is attached to open distal end (120), such that working element (101) may pass through bore (1550) as working element (101) is guided through distal open end (120). Thus, a working channel (1554) may extend along bore (1550).

Distal endoscope cap (1510) of the present example also includes an arch-shaped array of generally rectangular and generally cylindrical bores (1562a, 1562b) each extending longitudinally between proximal and distal surfaces (1544, 1546) of hub (1542) and disposed about (e.g., above) bore (1550). More particularly, distal endoscope cap (1510) includes an inner pair of laterally-opposed, generally rectangular bores (not shown) similar to bores (160) and an outer pair of laterally-opposed, generally cylindrical bores (1562a, 1562b). Distal endoscope cap (1510) further includes a pair of cameras (not shown) similar to cameras (164), which are each received within a respective inner bore in a manner similar to that described above; and a single illuminating element (1566) similar to illuminating elements (166), which is received within first outer bore (1562a) in a manner similar to that described above. In the example shown, illuminating element (1566) includes fiber optic components. More particularly, illuminating element (1566) is provided in the form of a lens that is optically coupled with at least one optical fiber (1569). Optical fiber (1569) may extend along shaft assembly (108) and be optically coupled with a source of light that is either integrated into handle assembly (106) (or some other body from which shaft assembly (108) extends) or otherwise provided.

As shown, distal endoscope cap (1510) of the present example also includes a single navigation sensor (1552), which is also received within first outer bore (1562a) and disposed (e.g., wrapped) about optical fiber (1569). In some versions, navigation sensor (1552) may be configured as a single-axis sensor. In some such cases, a plurality of (e.g., three) navigation sensors (1552) may be disposed linearly along optical fiber (1569) to collectively define a triple-axis sensor. In addition, or alternatively, navigation sensor (1552) may be similar to navigation sensor assembly (152).

In the example shown, distal endoscope cap (1510) further includes an optical sensor (1553), which is received within second outer bore (1562b). Optical sensor (1553) may be configured to function as a temperature sensor, a force sensor, and/or a pressure sensor. For example, optical sensor (1553) may be configured as a Fiber Bragg grating (FBG) sensor for detecting a temperature within an anatomical passageway of a patient. In addition, or alternatively, optical sensor (1553) may be configured for detecting a force applied to distal endoscope cap (1510) and/or a force applied by distal endoscope cap (1510) (e.g., against an anatomical structure of the patient). In some cases, a second navigation sensor (1552) may also be received within second outer bore (1562b) and disposed (e.g., wrapped) about optical sensor (1553).

While optical sensor (1553) is received within second outer bore (1562b) in the present example, it will be appreciated that various other types of components may be received within second outer bore (1562b) in addition to or in lieu of optical sensor (1553). For example, a second illuminating element (1566) may be received within second outer bore (1562b) and may be provided in the form of a lens that is optically coupled with at least one second optical fiber (1569). In some such cases, a second navigation sensor (1552) may also be received within second outer bore (1562b) and disposed (e.g., wrapped) about the second optical fiber (1569).

While not shown, distal endoscope cap (1510) of the present example may also include an irrigation lumen extending distally from proximal surface (1544) toward distal surface (1546) of hub (1542) and disposed above bore (1550) between the inner bores for directing streams of liquid generally laterally outwardly into the inner bores through fluid outlet slots (not shown) in a manner similar to that described above.

### VI. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a shaft assembly including a rigid proximal portion and a flexible distal portion, the flexible distal portion having an open distal end with a first outer diameter sized and configured to fit in an anatomical passageway in an ear, nose, or throat of a patient, the flexible distal portion being configured to enable lateral deflection of the distal end away from or toward a longitudinal axis defined by the rigid proximal portion, the shaft assembly defining a working channel sized and configured to enable advancement of a working element distally past the distal end of the shaft assembly; and (b) a distal endoscope cap configured to attach to the distal end of the shaft assembly, the distal endoscope cap having a second outer diameter, the second outer diameter being larger than the first outer diameter, the distal endoscope cap including: (i) a body having at least one coupling member for attaching the distal endoscope cap to the distal end of the shaft assembly, (ii) at least one image sensor secured to the body for visualizing an anatomical structure, and (iii) at least one illuminating element secured to the body for illuminating a field of view of the at least one image sensor.

### Example 2

The apparatus of Example 1, wherein the at least one image sensor includes a pair of image sensors.

### Example 3

The apparatus of any of Examples 1 through 2, wherein the at least one illuminating element includes a pair of illuminating elements.

### Example 4

The apparatus of Example 3, wherein each illuminating element of the pair of illuminating elements has a light intensity, wherein the light intensity of each illuminating element is independently adjustable.

### Example 5

The apparatus of Example 4, wherein the light intensity of each illuminating element is independently adjustable by a processor using an artificial intelligence program.

### Example 6

The apparatus of any of Examples 1 through 5, wherein the distal endoscope cap further includes an irrigation lumen configured to direct liquid toward the at least one image sensor.

### Example 7

The apparatus of Example 6, wherein the distal endoscope cap further includes a suction lumen configured to draw the liquid away from the at least one image sensor.

### Example 8

The apparatus of any of Examples 1 through 7, wherein the distal endoscope cap further includes a bore extending longitudinally through the body for axial alignment with the working channel of the shaft assembly to enable advancement of the working element from the working channel through the bore.

### Example 9

The apparatus of any of Examples 1 through 8, wherein the distal endoscope cap further includes at least one navigation sensor secured to the body, the at least one navigation sensor being configured to generate signals indicative of a position of the distal endoscope cap in three-dimensional space.

### Example 10

The apparatus of any of Examples 1 through 9, wherein the at least one image sensor is configured to distally extend and proximally retract relative to the body.

### Example 11

The apparatus of any of Examples 1 through 10, wherein the distal endoscope cap is operable to apply energy to tissue.

### Example 12

The apparatus of any of Examples 1 through 11, wherein the body includes a distal end, wherein the at least one image sensor is proximally recessed relative to the distal end.

### Example 13

The apparatus of any of Examples 1 through 12, wherein the at least one coupling member includes a pair of laterally-opposed wings configured to frictionally engage the distal end of the shaft assembly for attaching the distal endoscope cap to the distal end of the shaft assembly.

### Example 14

The apparatus of any of Examples 1 through 12, wherein the at least one coupling member includes a collar configured to frictionally engage the distal end of the shaft assembly for attaching the distal endoscope cap to the distal end of the shaft assembly.

### Example 15

The apparatus of any of Examples 1 through 14, wherein the distal endoscope cap is removably attached to the distal end of the shaft assembly.

### Example 16

A distal endoscope cap for providing visualization capabilities to a surgical instrument having a flexible shaft configured to be inserted into an anatomical passageway of a patient's head, the distal endoscope cap comprising: (a) a body having at least one coupling member for attaching the distal endoscope cap to a distal end of the flexible shaft, at least a portion of the body being configured to extend radially outwardly past an outer perimeter of the flexible shaft; (b) a bore extending longitudinally through the body for axial alignment with a working channel of the flexible shaft to enable advancement of a working element from the working channel through the bore; (c) at least one image sensor secured to the body for visualizing an anatomical structure; and (d) at least one illuminating element secured to the body for illuminating a field of view of the at least one image sensor.

### Example 17

The distal endoscope cap of Example 16, further comprising an irrigation lumen configured to direct liquid toward the at least one image sensor.

### Example 18

The distal endoscope cap of any of Examples 16 through 17, further comprising at least one navigation sensor secured to the body, the at least one navigation sensor being configured to generate signals indicative of a position of the distal endoscope cap in three-dimensional space.

### Example 19

The distal endoscope cap of any of Examples 16 through 18, wherein the at least one coupling member is configured to frictionally engage the distal end of the flexible shaft for attaching the distal endoscope cap to the distal end of the flexible shaft.

### Example 20

A method of providing visualization capabilities to a surgical instrument having a flexible shaft configured to be inserted into an anatomical passageway of a patient's head, the method comprising: (a) attaching a distal endoscope cap to a distal end of the flexible shaft such that a longitudinal bore of the distal endoscope cap is axially aligned with a working channel of the flexible shaft to enable advancement of a working element from the working channel through the bore, the act of attaching including securing a coupling member of the distal endoscope cap to a cylindrical outer surface of the flexible shaft; (b) routing at least one image sensor wire along the flexible shaft to at least one image sensor of the distal endoscope cap to enable visualization of an anatomical structure; and (c) routing at least one irrigation fluid conduit along the flexible shaft to at least one irrigation lumen of the distal endoscope cap to enable cleaning of the at least one image sensor.

### Example 21

An apparatus, comprising: (a) a body; (b) a shaft assembly extending distally from the body and defining a longitudinal axis, wherein the shaft assembly includes a flexible distal portion configured to be inserted into an anatomical passageway of a patient's head; and (c) a deflection actuation assembly, including: (i) a rotary input member configured to rotate relative to the body about the longitudinal axis, (ii) a linear output member slidably housed within the body and configured to translate relative to the body along the longitudinal axis in response to rotation of the rotary input member relative to the body about the longitudinal axis, wherein the linear output member is fixed against rotation relative to the body about the longitudinal axis, and (iii) an elongate actuation member extending through the shaft assembly, wherein the elongate actuation member is operatively coupled with each of the linear output member and the flexible distal portion, wherein the flexible distal portion is configured to deflect away from the longitudinal axis in response to translation of the linear output member relative to the body along the longitudinal axis.

### Example 22

The apparatus of Example 21, wherein the rotary input member includes one of a leadscrew or a lead nut, wherein the linear output member includes the other of a leadscrew or a lead nut.

### Example 23

The apparatus of Example 22, wherein the rotary input member includes the leadscrew, wherein the rotary output member includes the lead nut.

### Example 24

The apparatus of Example 23, wherein the rotary input member further includes a deflection control knob fixed against rotation relative to the leadscrew about the longitudinal axis.

### Example 25

The apparatus of Example 24, wherein the deflection control knob and the leadscrew are integrally formed together with each other as a unitary piece.

### Example 26

The apparatus of any of Example 21 through 25, further comprising at least one image sensor operatively coupled to the flexible distal portion for visualizing the anatomical passageway.

### Example 27

The apparatus of any of Examples 21 through 26, further comprising: (a) a distal suction tube extending through the shaft assembly; and (b) a proximal suction tube extending proximally from the body, wherein the proximal suction tube is in fluid communication with the distal suction tube, wherein the proximal suction tube is configured to rotate relative to the body about the longitudinal axis independently of the distal suction tube.

### Example 28

The apparatus of Example 27, further comprising a suction valve fluidly coupling the proximal suction tube to the distal suction tube.

### Example 29

The apparatus of Example 28, wherein at least one of the proximal suction tube or the distal suction tube is configured to rotate relative to the suction valve about the longitudinal axis.

### Example 30

The apparatus of any of Examples 21 through 29, further comprising at least one conduit extending through the shaft assembly.

### Example 31

The apparatus of Example 30, wherein the at least one conduit extends through the shaft assembly parallel to the longitudinal axis.

### Example 32

The apparatus of any of Examples 30 through 31, wherein the at least one conduit includes at least one of an irrigation fluid tube, an electrical wire, or an optical fiber.

### Example 33

The apparatus of any of Examples 30 through 32, wherein the rotary input member includes at least one slot, wherein the at least one conduit is routed through the at least one slot.

### Example 34

The apparatus of any of Examples 30 through 33, wherein the linear output member includes at least one slot, wherein the at least one conduit is routed through the at least one slot.

### Example 35

The apparatus of any of Examples 30 through 34, further comprising a strain relief configured to rotate relative to the body about the longitudinal axis independently of the rotary input member, wherein the strain relief is configured to direct the at least one conduit proximally away from the longitudinal axis.

### Example 36

An apparatus, comprising: (a) a body; (b) a shaft assembly extending distally from the body and defining a longitudinal axis, wherein the shaft assembly is configured to be inserted into an anatomical passageway of a patient's head; (c) at least one conduit extending through the shaft assembly, wherein the at least one conduit includes at least one of an irrigation fluid tube, an electrical wire, or an optical fiber; and (d) a strain relief configured to rotate relative to the body about the longitudinal axis, wherein the strain relief is configured to direct the at least one conduit proximally away from the longitudinal axis.

### Example 37

The apparatus of Example 36, wherein the at least one conduit extends through the shaft assembly parallel to the longitudinal axis.

### Example 38

The apparatus of any of Examples 36 through 37, wherein the strain relief is configured to direct the at least one conduit proximally away from the longitudinal axis along an exit axis that is oriented obliquely relative to the longitudinal axis.

### Example 39

The apparatus of any of Examples 36 through 38, wherein the strain relief includes: (i) a rigid strain relief body, and (ii) a flexible strain relief extension coupled to the rigid strain relief body.

### Example 40

An apparatus, comprising: (a) a body; (b) a shaft assembly extending distally from the body to a distal end and defining a longitudinal axis, wherein the distal end is configured to be inserted into an anatomical passageway of a patient's head; (c) at least one image sensor operatively coupled to the distal end of the shaft assembly for visualizing an anatomical structure; and (d) an irrigation dispensing assembly including: (i) a reservoir including a collapsible chamber configured to be pressurized with irrigation fluid, (ii) a valve configured to transition between an actuated state and an unactuated state, and (iii) an irrigation fluid conduit including: (A) an inlet port configured to be fluidly isolated from the collapsible chamber when the valve is in the unactuated state, and configured to be fluidly coupled with the collapsible chamber when the valve is in the actuated state for receiving the irrigation fluid therefrom, and (B) an outlet port configured to direct the irrigation fluid received from the collapsible chamber toward the at least one image sensor.

### Example 41

A distal endoscope cap for providing visualization capabilities to a surgical instrument having a flexible shaft configured to be inserted into an anatomical passageway of a patient's head, the distal endoscope cap comprising: (a) a body having at least one coupling member for attaching the distal endoscope cap to a distal end of the flexible shaft, at least a portion of the body being configured to extend radially outwardly past an outer perimeter of the flexible shaft; (b) a first bore extending longitudinally through the body for axial alignment with a working channel of the flexible shaft to enable advancement of a working element from the working channel through the first bore; (c) a second bore extending longitudinally at least partially through the body; (d) a navigation sensor secured to the body, wherein the navigation sensor is received within the second bore, the navigation sensor being configured to generate signals indicative of a position of the distal endoscope cap in three-dimensional space; (e) an image sensor secured to the body for visualizing an anatomical structure; and (f) an illuminating element secured to the body for illuminating a field of view of the image sensor.

### Example 42

The distal endoscope cap of Example 41, further comprising a third bore extending longitudinally at least partially through the body, wherein the illuminating element is received within the third bore.

### Example 43

The distal endoscope cap of Example 41, further comprising a third bore extending longitudinally at least partially through the body, wherein the illuminating element is received within the second bore.

### Example 44

The distal endoscope cap of any of Examples 41 through 43, further comprising a temperature sensor secured to the body for detecting a temperature within the anatomical passageway.

### Example 45

The distal endoscope cap of any of Examples 41 through 44, further comprising a force sensor secured to the body for detecting a force applied to the distal endoscope cap.

### Example 46

An apparatus comprising: (a) a flexible shaft, the flexible shaft being sized to fit in an anatomical passageway of a patient, the flexible shaft comprising: (i) a working channel, and (ii) a distal end; and (b) a distal body at the distal end of the flexible shaft, the distal body comprising: (i) a first bore extending longitudinally through the distal body for axial alignment with the working channel of the flexible shaft to enable advancement of a working element from the working channel through the first bore, (ii) a second bore extending longitudinally at least partially through the distal body, (iii) a navigation sensor secured to the distal body, wherein the navigation sensor is received within the second bore, the navigation sensor being configured to generate signals indicative of a position of the distal body in three-dimensional space, (iv) an image sensor secured to the distal body for visualizing an anatomical structure, and (v) an illuminating element secured to the distal body for illuminating a field of view of the image sensor.

### Example 47

The apparatus of Example 46, the distal body having at least one coupling member for attaching the distal body to the distal end of the flexible shaft

### Example 48

The apparatus of any of Examples 46 through 47, at least a portion of the distal body being configured to extend radially outwardly past an outer perimeter of the flexible shaft.

### Example 49

An apparatus comprising: (a) a shaft, the shaft being sized to fit in an anatomical passageway of a patient, the shaft comprising: (i) a working channel, and (ii) a distal end; and (b) a distal body at the distal end of the shaft, the distal body comprising: (i) a first bore extending longitudinally through the distal body, the first bore being sized and positioned relative to the working channel of the shaft to enable advancement of a working element from the working channel through the first bore, (ii) an image sensor secured to the distal body for visualizing an anatomical structure, (iii) an illuminating element secured to the distal body for illuminating a field of view of the image sensor, and (iv) a navigation sensor, the navigation sensor being configured to generate signals indicative of a position of the distal body in three-dimensional space, the navigation sensor being positioned coaxially about the illuminating element.

### Example 50

The apparatus of Example 49, the illuminating element including one or more optical fibers, the navigation sensor being positioned coaxially about the one or more optical fibers.

### VII. MISCELLANEOUS

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a shaft assembly including a rigid proximal portion and a flexible distal portion, the flexible distal portion having an open distal end with a first outer diameter sized and configured to fit in an anatomical passageway in an ear, nose, or throat of a patient, the flexible distal portion being configured to enable lateral deflection of the distal end away from or toward a longitudinal axis defined by the rigid proximal portion, the shaft assembly defining a working channel sized and configured to enable advancement of a working element distally past the distal end of the shaft assembly; and
(b) a distal endoscope cap configured to attach to the distal end of the shaft assembly, the distal endoscope cap having a second outer diameter, the second outer diameter being larger than the first outer diameter, the distal endoscope cap including:
(i) a body having at least one coupling member for attaching the distal endoscope cap to the distal end of the shaft assembly,
(ii) at least one image sensor secured to the body for visualizing an anatomical structure, and
(iii) at least one illuminating element secured to the body for illuminating a field of view of the at least one image sensor.

2. The apparatus of claim 1, the at least one image sensor including a pair of image sensors.

3. The apparatus of claim 1, the at least one illuminating element including a pair of illuminating elements.

4. The apparatus of claim 3, each illuminating element of the pair of illuminating elements having a light intensity, the light intensity of each illuminating element being independently adjustable.

5. The apparatus of claim 4, the light intensity of each illuminating element being independently adjustable by a processor using an artificial intelligence program.

6. The apparatus of claim 1, the distal endoscope cap further including an irrigation lumen configured to direct liquid toward the at least one image sensor.

7. The apparatus of claim 6, the distal endoscope cap further including a suction lumen configured to draw the liquid away from the at least one image sensor.

8. The apparatus of claim 1, the distal endoscope cap further including a bore extending longitudinally through the body for axial alignment with the working channel of the shaft assembly to enable advancement of the working element from the working channel through the bore.

9. The apparatus of claim 1, the distal endoscope cap further including at least one navigation sensor secured to the body, the at least one navigation sensor being configured to generate signals indicative of a position of the distal endoscope cap in three-dimensional space.

10. The apparatus of claim 1, the at least one image sensor being configured to distally extend and proximally retract relative to the body.

11. The apparatus of claim 1, the distal endoscope cap being operable to apply energy to tissue.

12. The apparatus of claim 1, the body including a distal end, the at least one image sensor being proximally recessed relative to the distal end.

13. The apparatus of claim 1, the at least one coupling member including a pair of laterally-opposed wings configured to frictionally engage the distal end of the shaft assembly for attaching the distal endoscope cap to the distal end of the shaft assembly.

14. The apparatus of claim 1, the at least one coupling member including a collar configured to frictionally engage the distal end of the shaft assembly for attaching the distal endoscope cap to the distal end of the shaft assembly.

15. The apparatus of claim 1, the distal endoscope cap being removably attached to the distal end of the shaft assembly.

16. An apparatus comprising:
(a) a shaft, the shaft being sized to fit in an anatomical passageway of a patient, the shaft comprising:
(i) a working channel, and
(ii) a distal end; and
(b) a distal body at the distal end of the shaft, the distal body comprising:
(i) a first bore extending longitudinally through the distal body, the first bore being sized and positioned relative to the working channel of the shaft to enable advancement of a working element from the working channel through the first bore,
(ii) an image sensor secured to the distal body for visualizing an anatomical structure,
(iii) an illuminating element secured to the distal body for illuminating a field of view of the image sensor, and
(iv) a navigation sensor, the navigation sensor being configured to generate signals indicative of a position of the distal body in three-dimensional space, the navigation sensor being positioned coaxially about the illuminating element.

17. The apparatus of claim 16, the illuminating element including one or more optical fibers, the navigation sensor being positioned coaxially about the one or more optical fibers.

18. An apparatus, comprising:
(a) a body;
(b) a shaft assembly extending distally from the body and defining a longitudinal axis, wherein the shaft assembly includes a flexible distal portion configured to be inserted into an anatomical passageway of a patient's head; and
(c) a deflection actuation assembly, including:
(i) a rotary input member configured to rotate relative to the body about the longitudinal axis,
(ii) a linear output member slidably housed within the body and configured to translate relative to the body along the longitudinal axis in response to rotation of the rotary input member relative to the body about the longitudinal axis, wherein the linear output member is fixed against rotation relative to the body about the longitudinal axis, and
(iii) an elongate actuation member extending through the shaft assembly, wherein the elongate actuation member is operatively coupled with each of the linear output member and the flexible distal portion, wherein the flexible distal portion is configured to deflect away from the longitudinal axis in response to translation of the linear output member relative to the body along the longitudinal axis.

19. The apparatus of claim 18, further comprising a strain relief configured to rotate relative to the body about the longitudinal axis independently of the rotary input member, wherein the strain relief is configured to direct the at least one conduit proximally away from the longitudinal axis.

20. The apparatus of claim 18, further comprising:
(a) at least one image sensor operatively coupled to a distal end of the shaft assembly for visualizing an anatomical structure; and
(b) an irrigation dispensing assembly including:
(i) a reservoir including a collapsible chamber configured to be pressurized with irrigation fluid,
(ii) a valve configured to transition between an actuated state and an unactuated state, and
(iii) an irrigation fluid conduit including:
(A) an inlet port configured to be fluidly isolated from the collapsible chamber when the valve is in the unactuated state, and configured to be fluidly coupled with the collapsible chamber when the valve is in the actuated state for receiving the irrigation fluid therefrom, and
(B) an outlet port configured to direct the irrigation fluid received from the collapsible chamber toward the at least one image sensor.
